(19) United States
(12) Patent Application Publication
(10) Pub. No.: US 2008/0207885 A1
(43) Pub. Date: Aug. 28, 2008

(54) Method for site-specific labeling of RNA using a deoxyribozyme

(76) Inventors: Scott K. Silverman, Savoy, IL (US); Dana A. Baum, Savoy, IL (US); Claudia Hoebartner, Goettingen (DE)

Correspondence Address: Licata & Tyrrell P.C., 66 E. Main Street, Marlton, NJ, 08053, US

(21) Appl. No.: 12/029,635

(22) Filed: Feb. 12, 2008

Related U.S. Application Data
(60) Provisional application No. 60/891,278, filed on Feb. 23, 2007.
(60) Provisional application No. 60/975,588, filed on Sep. 27, 2007.

(51) Int. Cl. C07H 21/04

(52) U.S. Cl. 536/23.1

(57) ABSTRACT

The present invention is a method for site-specific internal RNA modification. In accordance with the present method, a deoxyribozyme (DNA enzyme) is used as a catalyst to attach a tagging RNA to a pre-determined internal position of a target RNA molecule, wherein the tagging RNA is coupled to a label prior to or after attachment to the target RNA molecule thereby labeling the target RNA.

### INTRODUCTION

This application claims benefit of priority to U.S. Provisional Patent Application Ser. Nos. 60/891,278, filed Feb. 23, 2007, and 60/975,588, filed Sep. 27, 2007, the contents of which are incorporated herein by reference in their entireties.

This invention was made in the course of research sponsored by the National Institutes of Health (NIH Grant Nos. R01 GM065966 and F32 GM079036). The U.S. government has certain rights in this invention.

### BACKGROUND OF THE INVENTION

Site-specific covalent modification of RNA is important for enabling structure-function studies. For example, probes such as fluorescein are commonly used in fluorescence resonance energy transfer (FRET) investigations of RNA folding (Lilley (2004) RNA 10:151-158; Lemay, et al. (2006) Chem. Biol. 13:857-868; Ha (2004) Biochemistry 43:4055-4063; Bokinsky and Zhuang (2005) Acc. Chem. Res. 38:566-573; Bokinsky, et al. (2003) Proc. Natl. Acad. Sci. USA 100:9302-9307). Biotin is used for immobilization during single-molecule analysis (Lilley (2004) supra; Lemay, et al. (2006) supra; Ha (2004) supra; Bokinsky and Zhuang (2005) supra; Bokinsky, et al. (2003) supra), to enable RNA-protein crosslinking studies (Rhode, et al. (2003) RNA 9:1542-1551), and as a key element of in vitro selection schemes (Joyce (2004) Annu. Rev. Biochem. 73:791-836). The 5′- and 3′-termini of RNA may be derivatized (Odom, Jr., et al. (1980) Biochemistry 19:5947-5954), but many experiments instead demand internal modification, and no direct methods are conventionally available for site-specific modification within an arbitrary RNA sequence. Therefore, covalent modifications are typically introduced by enzymatic splint ligation (Moore and Sharp (1992) Science 256:992-997; Moore and Query (2000) Methods Enzymol. 317:109-123), in which a DNA template aligns oligoribonucleotide substrates that have modified nucleotides incorporated via solid-phase synthesis (Rhode, et al. (2003) supra; Klostermeier and Millar (2001) Biopolymers 61:159-179; Strobel and Ortoleva-Donnelly (1999) Chem. Biol. 6:153-165; Kurschat, et al. (2005) RNA 11:1909-1914; Hougland, et al. (2005) PLoS Biol. 3:e277; Höbartner, et al. (2005) J. Am. Chem. Soc. 127:12035-12045; Rhode, et al. (2006) EMBO J. 25:2475-2486). However, this approach often suffers from low yields and is unpredictable because identifying a high-yielding ligation site in the target RNA can be difficult without directly testing several possibilities. Unnatural nucleotides have also been used to transcribe modified RNAs (Kawai, et al. (2005) J. Am. Chem. Soc. 127:17286-17295; Moriyama, et al. (2005) Nucleic Acids Res. 33:e129; Hirao, et al. (2006) Nat. Methods 3:729-735; Hirao (2006) BioTechniques 40:711-717). While this avoids the difficulties of splint ligation, extensive organic synthesis is required. As an alternative approach for RNA labeling, noncovalent Watson-Crick hybridization of a probe-labeled oligonucleotide has been used (Mergny, et al. (1994) Nucleic Acids Res 22:920-928; Okamura, et al. (2000) Nucleic Acids Res. 28:e107; Tsuji, et al. (2001) Biophys. J. 81:501-515; Dorywalska, et al. (2005) Nucleic Acids Res. 33:182-189; Smith, et al. (2005) RNA 11:234-239; Robertson, et al. (2006) Biochemistry 45:6066-6074). However, this is invasive because long stretches of nucleotides must be inserted within the RNA, and duplex formation involving these inserted nucleotides must be tolerated. Accordingly, there is a need in the art for an efficient method for RNA labeling at an internal site.

### SUMMARY OF THE INVENTION

The present invention relates to methods for labeling a target ribonucleic acid (RNA) molecule. In one embodiment, the method involves contacting a target RNA with a tagging RNA in the presence of a deoxyribozyme that is complementary to at least a portion of the target RNA and at least a portion of the tagging RNA so that the tagging RNA is site-specifically attached to the target RNA, wherein the tagging RNA is coupled to a label prior to or after attachment to the target RNA thereby labeling the target RNA molecule. In accordance with this embodiment, the method further includes the step of contacting the labeled target RNA with a second deoxyribozyme to remove one or more tagging RNA nucleotides. In an alternative embodiment, the method involves contacting a target RNA with at least one phosphorylated nucleotide in the presence of a cofactor and deoxyribozyme that is complementary to at least a portion of the target RNA, the phosphorylated nucleotide and at least a portion of the cofactor so that the phosphorylated nucleotide is site-specifically attached to the target RNA. In accordance with this embodiment, the phosphorylated nucleotide can be coupled with a label prior to or after being attached to the target RNA.

### BRIEF DESCRIPTION OF THE DRAWINGS

FIG. 1 depicts deoxyribozyme-catalyzed labeling (DECAL) of RNA. FIG. 1A shows the coupling of the amine-reactive form of the label (filled circle) to 5-aminoallylcytidine, which was incorporated into the 19-nt tagging RNA by in vitro transcription. FIG. 1B shows labeling of the target RNA. The 2′-OH of a specific adenosine of the target RNA attacks the 5′-triphosphate of the labeled tagging RNA. FIG. 1C shows testing of four L substrates with the unmodified tagging RNA. FIGS. 1D-1H show testing the four L substrates with the tagging RNA modified either with a 5-aminoallyl-C (FIG. 1D) at the second position or with the biotin (FIG. 1E), DABCYL (FIG. 1F), fluorescein (FIG. 1G) or TAMRA (FIG. 1H) appended to the aminoallyl group. Circles, parent; squares, transversions-1; diamonds, transversions-2; and triangles, transitions.

FIG. 2 shows the generality of deoxyribozyme-catalyzed RNA labeling using the 10DM24 deoxyribozyme and the P4-P6 RNA. FIG. 2A shows the secondary structure of P4-P6 (SEQ ID NO:3). The ten tested adenosines are boxed. FIG. 2B shows the labeling yields after 2 hours. The modification to each tagging RNA is indicated in the legend.

FIG. 3 shows native PAGE data for unmodified (circles) and doubly labeled (triangles) P4-P6 RNA, showing almost no shift in [Mg²⁺]_{1/2} due to appending the labels. The slight reduction in the limiting high-Mg²⁺ relative mobility was as expected from the experiments with DNA-modified P4-P6 (in particular, the control experiments in which two noncomplementary DNA strands were attached to P4-P6 as described in Miduturu and Silverman (2005) J. Am. Chem. Soc. 127:10144-10145).

FIG. 4 is a schematic showing the truncation of the tagging RNA by the 10-23 deoxyribozyme.

FIG. 5 shows the Mg²⁺-dependence of FRET efficiency (E_{FRET}) for wild-type P4-P6 (circles), the nonfoldable mutant (triangles), and the tetraloop mutant (diamonds). E_{FRET} was determined by the (ratio)_{A} method (Clegg (1992) Methods Enzymol. 211;353-388; Lilley (2000) Methods Enzymol. 317:368-393).

FIG. 6 shows the sequences and proposed secondary-structure of several RNA-cleaving deoxyribozymes. FIG. 6A (SEQ ID NO:6) and FIG. 6B (SEQ ID NO:7) show deoxyribozymes selected using Mg²⁺ or Pb²⁺ as cofactor (Breaker and Joyce (1994) Chem. Biol. 1:223-229; Breaker and Joyce (1995) Chem. Biol. 2:655-660). FIG. 6C (SEQ ID NO:8) and FIG. 6D (SEQ ID NO:9), respectively show the 10-23 and the 8-17 deoxyribozymes selected in Mg²⁺ to cleave all-RNA substrates (Santoro and Joyce (1997) Proc. Natl. Acad Sci. USA 94:4262-4266). FIG. 6E (SEQ ID NO:10) depicts a deoxyribozyme selected using L-histidine as cofactor. FIG. 6F (SEQ ID NO:11) shows the 17E deoxyribozyme selected in Zn²⁺. In each structure, the upper strand is the substrate and the lower strand is the enzyme. Arrows identify the site of RNA transesterification.

FIG. 7 shows the 10DM24 deoxyribozyme and use of a small-molecule substrate. FIG. 7A shows the secondary structure and schematic three-helix-junction tertiary structure of 10DM24 (SEQ ID NO:12) in Watson-Crick base pairing with a target RNA having branch-site adenosine A (SEQ ID NO:13) and tagging RNA containing a 5′ triphosphorylated nucleotide (SEQ ID NO:14). The 5′-triphosphorylated guanosine electrophile is presented to the branch-site adenosine nucleophile while held at the terminus of the P4 (paired region P4) RNA:DNA helix by Watson-Crick hydrogen bonds. Conceptually breaking the right-hand (R) oligonucleotide substrate (i.e., the tagging RNA) immediately to the 3′-side of its first nucleotide leads in principle to a deoxyribozyme substrate complex in which guanosine 5′-triphosphate (GTP) can bind as a discrete electrophile in the location corresponding to the 5′-terminal position of the P4 helix (FIG. 7B).

FIG. 8 shows the reaction of a small-molecule NTP substrate catalyzed by the 10DM24 deoxyribozyme. Successful ligation was observed only when the NTP substrate had Watson-Crick complementarity to the terminal P4 DNA nucleotide of 10DM24. FIG. 8A depicts Watson-Crick interactions between the NTP substrate (top) and the terminal P4 DNA nucleotide of 10DM24 (bottom). FIG. 8B shows kinetic plots for Watson-Crick combinations. The solid lines denote reactions of NTPs that form three Watson-Crick hydrogen bonds with the deoxyribozyme, whereas the dashed lines denote reactions of NTPs that form only two Watson-Crick hydrogen bonds.

FIG. 9 shows the assessment of potential stacking interactions that involve the NTP substrate. The ligation reactions were performed under standard incubation conditions.

FIG. 10 depicts the use of a second NTP as a cofactor for the ligation reaction.

FIG. 11 shows the 10DM24-catalyzed ligation of pppGpG. Reactions were performed at 1 mM pppGpG and 40 mM MgCl₂ in 100 mM CHES, pH 9.0, 150 mM NaCl, and 2 mM KCl at 37° C. k_{obs} values are indicated.

FIG. 12 shows the dependence of k_{obs} on the concentration of pppGpG and determination of K_{d,app} for pppGpG at 40 mM Mg²⁺ in 100 mM CHES, pH 9.0, 150 mM NaCl, and 2 mM KCl at 37° C.

### DETAILED DESCRIPTION OF THE INVENTION

The present invention is a novel method for site-specific internal RNA modification. In accordance with the present method, a deoxyribozyme (DNA enzyme) is used as a catalyst to attach a short tagging RNA to a pre-determined internal position of a target RNA molecule. RNA labeling of the present invention is said to be site-specific in that the label is attached at a particular pre-determined position along the RNA chain. This contrasts with random labeling, in which one or more labels are attached indiscriminately to the RNA. Moreover, unlike conventional methods for site-specific labeling of large target RNAs, the instant method, referred to herein as deoxyribozyme-catalyzed labeling (DECAL) of RNA, does not require solid-phase synthesis and labeling of a small RNA fragment and then assembly of the large target RNA by one or more RNA ligation reactions. Because such ligation reactions often proceed poorly and must be optimized carefully, a method that avoids such reactions entirely is highly desirable. In addition, the present method can be carried out without multistep organic synthesis of complicated precursor compounds.

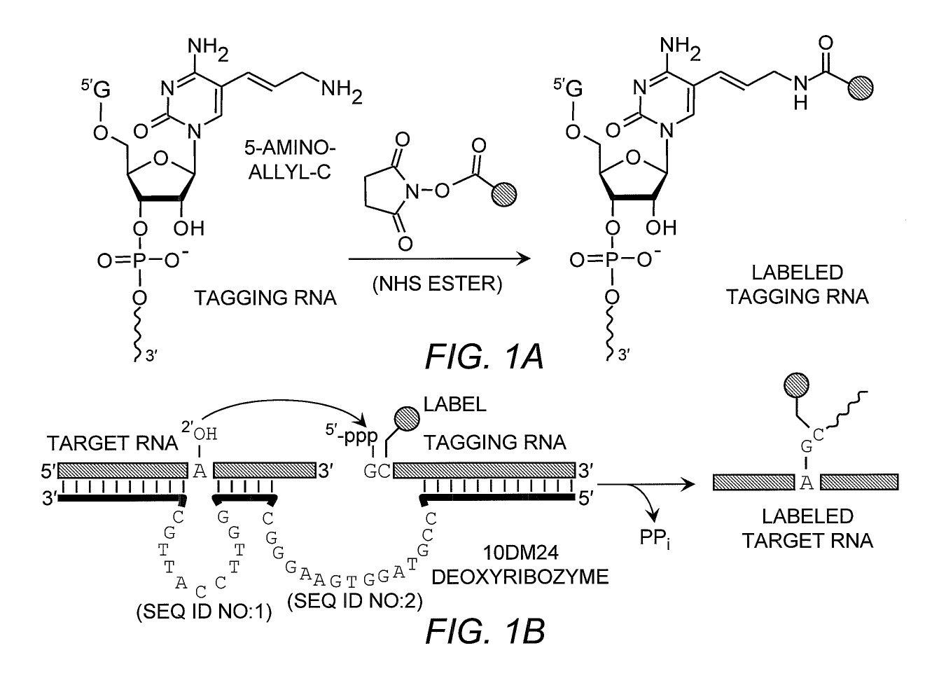

By way of example, a single 5-aminoallylcytidine nucleotide was incorporated at the second nucleotide position of a short “tagging RNA” by in vitro transcription. The aminoallyl-modified transcript was coupled with the amine-reactive form of a desired biophysical label to form a labeled tagging RNA (FIG. 1A). The tagging RNA was then attached by the deoxyribozyme to an internal 2′-hydroxyl of the target RNA (FIG. 1B) This RNA modification approach avoids solid-phase synthesis because modified nucleotides such as 5-aminoallylcytidine nucleotide triphosphate necessary for in vitro transcription of the tagging RNA are commercially available. This RNA modification approach also avoids organic synthesis because labeling of the tagging RNA requires only commercially available reagents and biochemical purification steps (e.g., PAGE). Furthermore, because the intact target RNA is derivatized directly with the label, splint ligation is entirely obviated, and no mutations are required in the target RNA to provide a modification site.

It has been shown that the 10DM24 deoxyribozyme has considerable sequence tolerance with respect to its RNA substrates (Zelin, et al. (2006) Biochemistry 45:2767-2771). Therefore, to illustrate the inventive method, analysis was carried out to test the ability of 10DM24 to use a tagging RNA derivatized with the representative biophysical labels biotin, DABCYL (a quencher), fluorescein, or TAMRA (tetramethylrhodamine). 10DM24 catalyzed label attachment to a comprehensive set of short target RNA substrates with general applicability (FIGS. 1C-1H).

To implement the DECAL strategy with a large RNA, ten sites within the 160-nucleotide Tetrahymena group I intron P4-P6 domain (Murphy and Cech (1993) Biochemistry 32;5291-5300; Murphy and Cech (1994) J. Mol. Biol. 236:49-63) were selected. This RNA was employed as it is routinely used as a model RNA (Murphy and Cech (1993) supra; Murphy and Cech (1994) supra; Cate, et al. (1996) Science 273:1678-1685; Silverman and Cech (1999) Biochemistry 38:8691-8702; Silverman and Cech (1999) Biochemistry 38:14224-14237; Smalley and Silverman (2006) Nucleic Acids Res. 34:152-166; Young and Silverman (2002) Biochemistry 41:12271-12276; Basu, et al. (1998) Nat. Struct. Biol. 5:927-930; Juneau and Cech (1999) RNA 5:1119-1129; Schwans, et al. (2003) J. Am. Chem. Soc. 125:10012-10018; Schwans, et al. (2004) Angew. Chem. 116:3095-3099; Schwans, et al. (2004) Angew. Chem. Int. Ed. 43:3033-3037; Yoshioka, et al. (2004) RNA 10:1900-1906; Das, et al. (2005) J. Am. Chem. Soc. 127:8272-8273). Target sites were selected on the basis of 2′-OH accessibility of adenosines in the X-ray crystal structure (Cate, et al. (1996) supra) because 10DM24 prefers adenosine 2′-OH groups (Zelin, et al. (2006) supra). Specifically included were target sites that would be useful in FRET studies if they were successfully derivatized. P4-P6 tagging was tested with a tagging RNA that lacked the aminoallyl group, as well as with tags incorporating aminoallyl, biotin, fluorescein, and TAMRA.

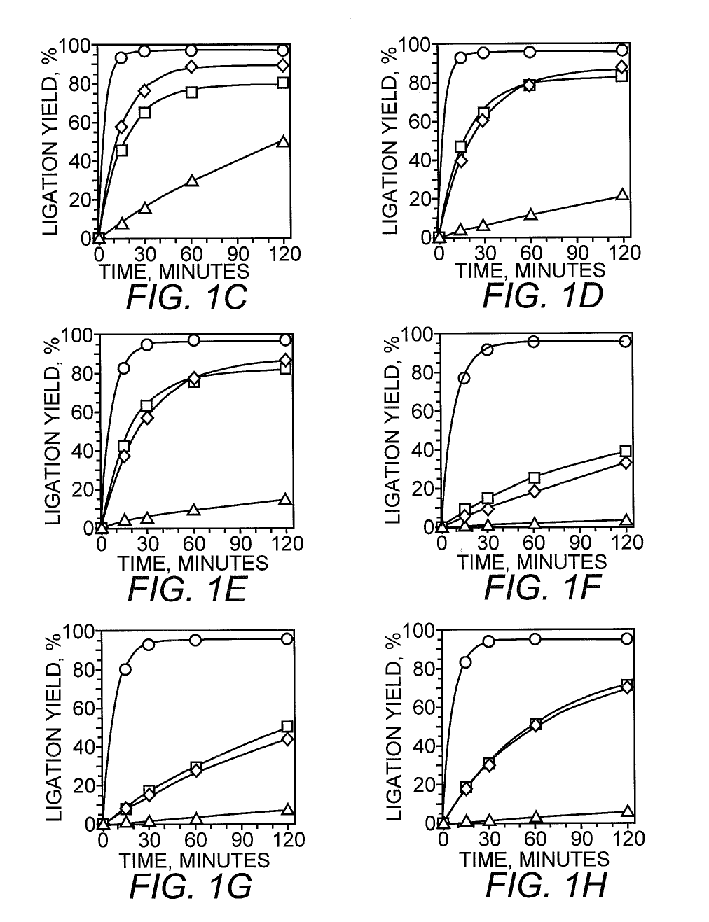

Six of the ten tested P4-P6 sites were derivatized in >50% yield using a tagging RNA that had only the aminoallyl modification (FIG. 2). The same six locations were labeled with biotin in >40% yield. The fluorescein label was appended to five sites with >40% yield, while the TAMRA label was attached at one location with >50% yield. On the basis of these results, two sites (A231 and A146) were chosen for preparative labeling of P4-P6 with the FRET pair fluorescein and TAMRA. The fluorescein label was attached to A231, and the singly labeled product was purified by PAGE. The TAMRA label was then appended to A146, leading to the doubly labeled P4-P6. Because of the gel shift upon each label addition, the final PAGE-purified product was homogenous with respect to the two labels.

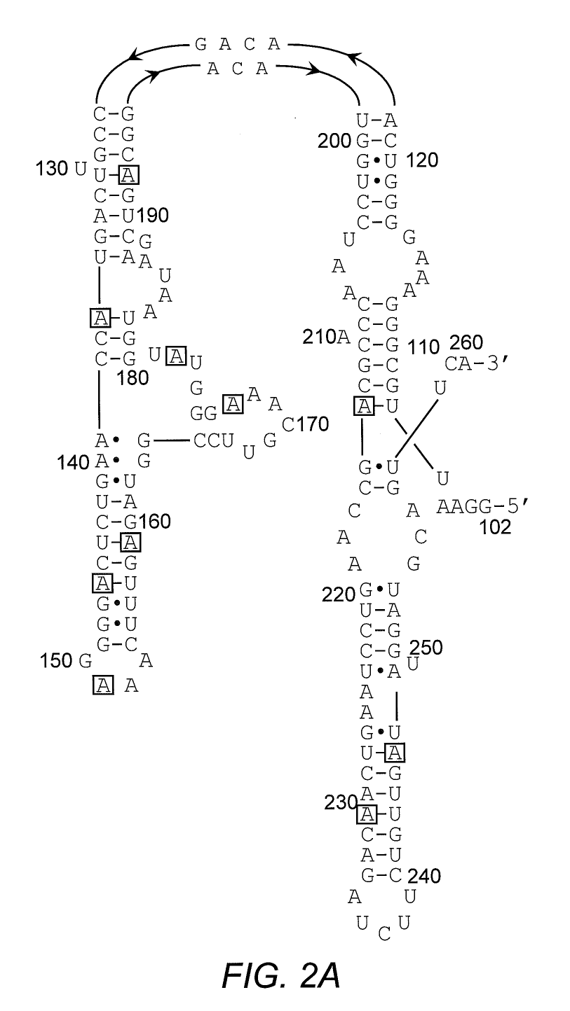

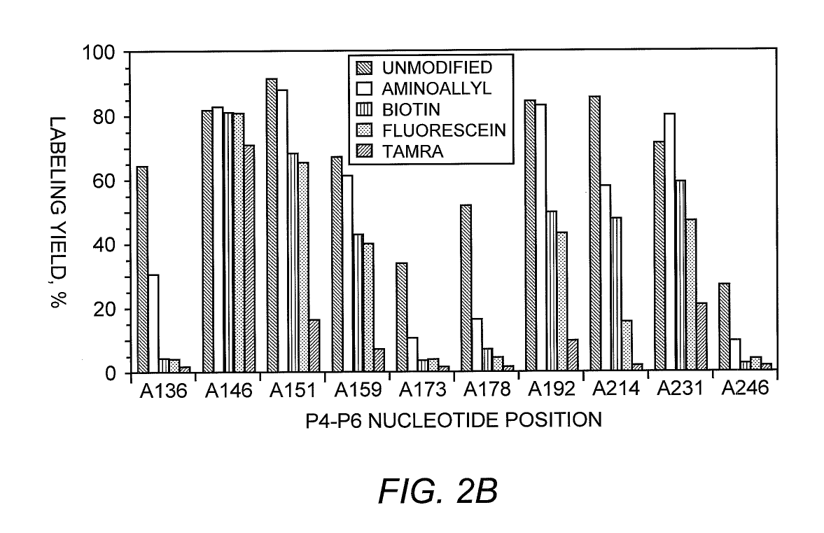

Both A231 and A146 are part of canonical helical regions and not involved in tertiary interactions. Therefore, no perturbation of the native P4-P6 RNA folding was expected upon attachment of the two labeled tagging RNAs. To verify this experimentally, Mg²⁺-dependent folding was assayed by non-denaturing PAGE (Silverman and Cech (1999) Biochemistry 38:8691-8702; Silverman and Cech (1999) Biochemistry 38:14224-14237; Smalley and Silverman (2006) supra; Young and Silverman (2002) supra). Attachment of the fluorescein and TAMRA labels to P4-P6 caused almost no shift in Mg²⁺-dependence (FIG. 3; ΔΔG°=0.5 kcal/mol). Although the labels do not perturb folding of P4-P6, other RNA targets could be more sensitive. To address this, the 10-23 deoxyribozyme (Santoro and Joyce (1997) supra) (FIG. 4) was used to truncate each tagging RNA efficiently, leaving only eight tag nucleotides at each labeling site. However, incorporation of one or more phosphorothioates into the tagging RNA did not permit cleavage of the majority of the tagging RNA in preparatively useful fashion after labeling of the target RNA.

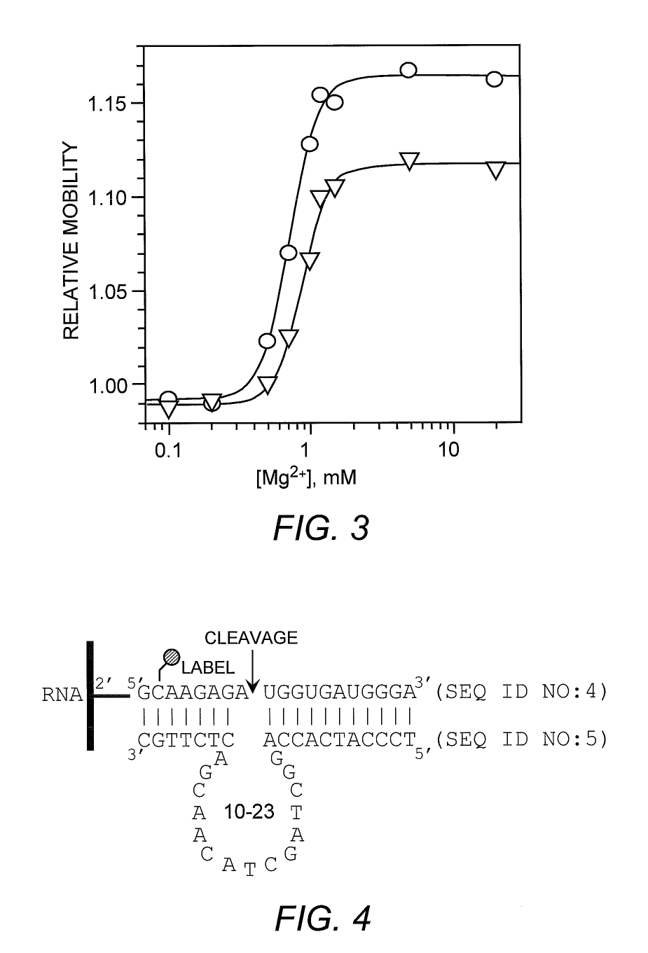

The Mg²⁺-dependent folding of doubly tagged P4-P6 was investigated by steady-state FRET. When P4-P6 was unfolded (at low Mg²⁺), the labeled A231 and A146 sites were relatively far apart due to opening of the “hinge” region, and the observed FRET efficiency (E_{FRET}) was ˜0 (FIG. 5). When the Mg²⁺ concentration is raised, folding of the RNA brings the two tagged sites closer together (Murphy and Cech (1993) supra; Murphy and Cech (1994) supra; Cate, et al. (1996) supra), which is expected to increase E_{FRET}. In addition to wild-type P4-P6 (P4-P6-wt), two mutant forms of P4-P6 were each doubly labeled with fluorescein and TAMRA. “Nonfoldable” P4-P6 (P4-P6-bp) contains base pairs in the hinge that disrupt folding (Murphy and Cech (1993) supra; Murphy and Cech (1994) supra; Silverman and Cech (1999) supra; Szewczak and Cech (1997) RNA 3:838-849). The second P4-P6 mutant had two adenosines inserted into the tetraloop, which was previously shown to increase the Mg²⁺-dependence considerably (Young and Silverman (2002) supra).

E_{FRET} was observed to increase at higher Mg²⁺ for the doubly labeled P4-P6-wt, with [Mg²⁺]_{1/2} of 1.6 mM (FIG. 5). P4-P6-bp had essentially no change in E_{FRET} at low Mg²⁺ (<10 mM). At higher Mg²⁺ the E_{FRET} increased, indicating that the fluorophores could come closer together due to RNA folding or compaction. Also, the tetraloop mutant P4-P6 had its [Mg²⁺]_{1/2} shifted considerably to the right. The higher E_{FRET} observed for the tetraloop mutant at >10 mM Mg²⁺ indicated a folded structure that allowed the two fluorophores to come closer together than in P4-P6-wt. The E_{FRET} values for all three P4-P6 RNAs were similar (˜0) at very low Mg²⁺, indicating similar unfolded states. FRET provides information about P4-P6 folding that cannot be obtained by native PAGE or single-fluorophore methods (Silverman and Cech (1999) supra; Silverman and Cech (1999) supra; Smalley and Silverman (2006) surpa; Young and Silverman (2002) surpa), thereby demonstrating the utility of the DECAL approach in labeling RNA molecules for FRET analysis.

Having demonstrated the use of deoxyribozymes for site-specific labeling of a target RNA, the present invention is a method for labeling a target RNA molecule. The method involves contacting a target RNA with the tagging RNA in the presence of a deoxyribozyme that is complementary to at least a portion of the target RNA and tagging RNA so that the tagging RNA is site-specifically attached to the target RNA. Advantageously, the attached tagging RNA is coupled to a label either prior to or after attachment to the target RNA to generate a labeled target RNA molecule.

In accordance with the present invention, a “target RNA” refers to any RNA molecule including, but not limited to, mRNA, tRNA, hnRNA, rRNA, a catalytic RNA, and the like. The target RNA can be, for example, cellular RNA or it can be an RNA containing a sequence that is the same as or complementary to a sequence of a cellular RNA. For example, the target RNA can be a product of in vivo or in vitro transcription of a gene of interest or a portion of such a gene.

Generally, RNA molecules of the invention are composed of nucleosides (ribose sugars with attached nucleobases) coupled by phosphodiester bonds. An RNA molecule of the present invention can also be an RNA:DNA hybrid or chimera, wherein the RNA portion of the hybrid is desirably labeled. Naturally-occurring DNA and RNA have natural bases such as adenosine (A), guanosine (G), thymidine (T), cytidine (C), and uridine (U). According to the Watson and Crick rules of base pairing, the natural bases can hybridize to form purine-pyrimidine base pairs, where G pairs with C and A pairs with T or U. These pairing rules facilitate specific hybridization of a nucleic acid molecule with a complementary nucleic acid molecule.

For the purposes of the present invention, a tagging RNA is defined as an RNA molecule (e.g., 1 to 30 nucleotides in length), which has been derivatized by substitution at one or more non-hydrogen bonding sites to form modified natural bases. For example, a natural base can be derivatized by coupling a reactive functional group to a non-hydrogen bonding atom of the base. Examples of suitable functional groups include, but are not limited to, amines, thiols, hydrazines, alcohols or alkyl groups or any other group typically used by the skilled artisan. In this regard, the tagging RNA has one or more functional groups which exhibit some chemical reactivity. In some embodiments, the RNA is aminoallyl-modified.

Either prior to or after attachment to the target RNA molecule, the tagging RNA molecule is coupled to a label. In certain embodiments, the tagging RNA is labeled at the 5′ end, e.g., at one or more of the nucleotides located at positions 1, 2, 3, 4, 5, 6, 7 or 8 relative to the 5′ end of the tagging RNA. A label of the present invention can be any small molecule, natural product, non-natural polymer, functional group or solid-phase bound tether. Desirably, the label of the present invention is in a form which is reactive (e.g., amine-reactive) with the one or more functional groups of the tagging RNA molecule thereby facilitating the coupling of the label to the tagging RNA. Examples of small molecules include, without limitation, biotin and fluorescein or any other detectable reporter molecule. Examples of natural products include, without limitation, peptides, proteins, nucleic acids, and carbohydrates including members of a specific binding pair (e.g., a ligand/receptor or antigen/antibody pair). Peptoids are an example of a non-natural polymer label (Zuckermann, et al. (1992) J. Am. Chem. Soc. 114:10646). Examples of useful functional groups include those disclosed herein, as well as functional groups with reactivities orthogonal to the reactivities of, e.g., protein functional groups (e.g., double bonds and ketones). In another embodiment of the invention, the label can be a tether linked to a solid phase. Such labels enable the ready attachment of target RNA molecules to columns, beads, or chip surfaces.

Labels particularly embraced by the present invention include, but are not limited to, biotin; fluorescent molecules such as umbelliferone, fluorescein, fluorescein isothiocyanate, rhodamine, dichlorotriazinylamine fluorescein, dansyl chloride or phycoerythrin; chemiluminescent molecules; digoxigenin; spin labels; radiolabels; and chromophores.

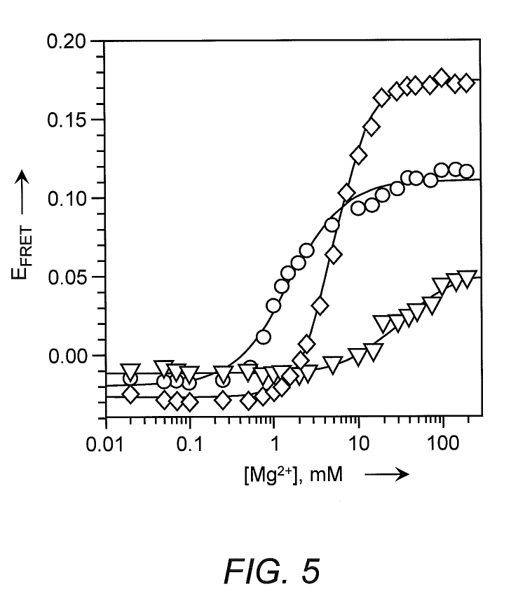

As shown in FIG. 1, the target RNA is contacted with the labeled tagging RNA in the presence of a deoxyribozyme that is complementary to at least a portion of the target RNA and labeled tagging RNA so that the target RNA is labeled. For the purposes of the present invention, deoxyribozymes are catalytic DNAs which have been identified by in vitro selection (Silverman (2004) Org. Biomol. Chem. 2:2701-2706; Peracchi (2005) ChemBioChem 6:1316-1322; Silverman (2005) Nucleic Acids Res 33:6151-6163). Several deoxyribozymes that ligate two RNA substrates have been identified (Flynn-Charlebois, et al. (2003) J. Am. Chem. Soc. 125:2444-2454; Purtha, et al. (2005) J. Am. Chem. Soc. 127:13124-13125; Wang and Silverman (2005) Angew. Chem. 117:6013-6016; Wang and Silverman (2005) Angew. Chem. Int. Ed. 44:5863-5866; Pratico, et al. (2005) Nucleic Acids Res. 33:3503-3512; Coppins and Silverman (2005) Biochemistry 44:13439-13446) and there are a number of deoxyribozymes which have been discovered or developed showing a great diversity in catalytic activity (see Table 1). While the present disclosure illustrates the use of the 10DM24 (SEQ ID NOs:1 and 2) and 10-23 (SEQ ID NO:8) deoxyribozymes, the skilled artisan can appreciate that any suitable deoxyribozyme can be employed for use in accordance with the present invention. Examples of such deoxyribozymes include the deoxyribozymes shown in FIGS. 6A-6F, Table 1 and deoxyribozymes with extended chemical functionality (Santoro, et al. (2000) J. Am. Chem. Soc. 122:2433-2439).

TABLE 1

| Reaction | Cofactor | Reference |
|---|---|---|
| RNA transester. | Pb²⁺ | Breaker and Joyce (1994) supra |
| | Mg²⁺ | Breaker and Joyce (1995) supra |
| | Ca²⁺ | Faulhammer, et al. (1997) J. Mol. Biol. |
| | | 269: 188 |
| | Mg²⁺ | Santoro and Joyce (1997) supra |
| | None | Geyer, et al. (1997) Chem. Biol. 4: 579 |
| | L-histidine | Roth and Breaker (1998) PNAS 95: 6027 |
| | Zn²⁺ | Li (2000) Nucl. Acids Res. 28: 481 |
| DNA cleavage | Cu²⁺ | Carmi, et al. (1996) Chem. Biol. 3: 1039 |
| DNA ligation | Cu²⁺ or | Cuenoud and Szostak (1995) Nature |
| | Zn²⁺ | 375: 611 |
| DNA | Ca²⁺ | Li and Breaker (1999) PNAS 96: 2746 |
| phosphorylation | | |
| 5,5′- | Cu²⁺ | Li, et al. (2000) Biochemistry 39: 3106 |
| pyrophosphate | | |
| formation | | |
| Porphyrin | None | Li and Sen (1996) Nat. Struct. Biol. |
| metalation | | 3: 743 |

Alternatively, the deoxyribozyme can be produced by in vitro selection in which DNA molecules with certain functions are isolated from a large number of sequence variants through multiple cycles of selection and amplification (Joyce (1994) Curr. Opin. Struct. Biol. 4:331-336; Chapman and Szostak (1994) Curr. Opin. Struct. Biol. 4:618-622). In vitro selection is typically initiated with a large collection of randomized sequences. A typical DNA library for selection contains 10¹³-10¹⁶ sequence variants. The construction of a completely randomized pool is accomplished by chemical synthesis of a set of degenerate oligonucleotides using standard phosphoramidite chemistry. The 3′-phosphoramidite compounds of four nucleosides (A, C, G, and T) are premixed before being supplied to an automated DNA synthesizer to produce oligonucleotides. By controlling the ratio of four phosphoroamidites, the identity at each nucleotide position can be either completely random, i.e., with equal chance for each base, or biased toward a single base. Other strategies for creating a randomized DNA library include applying mutagenic polymerase chain reaction (PCR) and template-directed mutagenesis (Tsang and Joyce (1996) Methods Enzymol. 267:410-426; Cadwell and Joyce (1994) PCR Methods Appl. 3:S136-S140).

In vitro selection takes advantage of a unique property of DNA, i.e., the same molecule can possess both genotype (coding information) and phenotype (encoded function). The DNA molecules in the randomized library are screened simultaneously. Those sequences that exhibit a desired function (phenotype) are separated from the inactive molecules. Usually the separation is performed through affinity column chromatography, being linked to or released from a solid support, gel electrophoresis separation, or selective amplification of a tagged reaction intermediate. The genotypes of the active molecules are then copied and amplified, normally through polymerase chain reaction (PCR) for DNA. Mutations can be performed with mutagenic PCR to reintroduce diversity to the evolving system. These three steps of selection, amplification and mutation, are repeated, often with increasing selection stringency, until sequences with the desired activity dominate the pool.

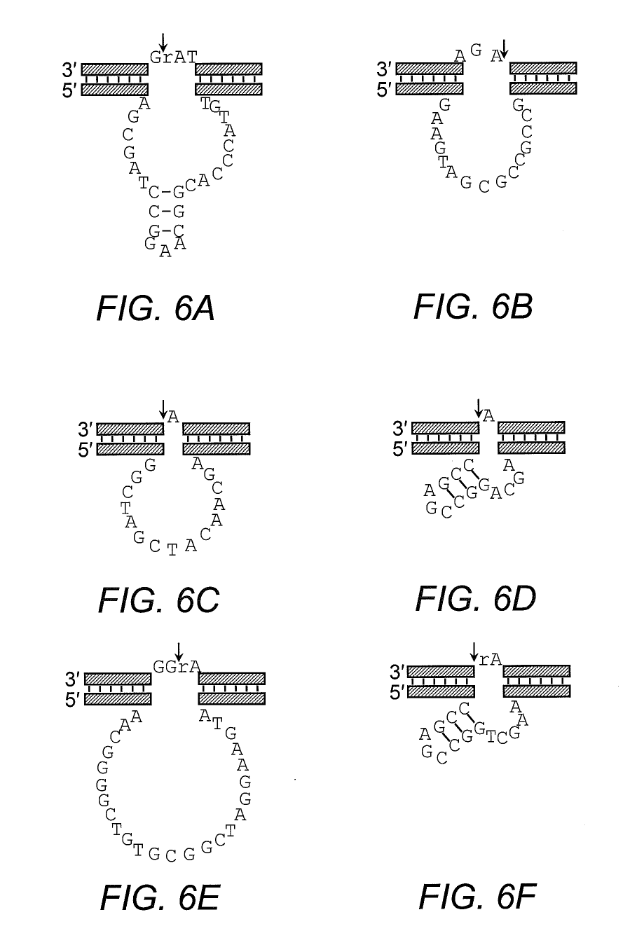

Independent of the deoxyribozyme employed, at least a first portion of the deoxyribozyme (e.g., one arm) is complementary to a portion of the tagging RNA and a second portion of the deoxyribozyme (e.g., the other arm) is complementary to a portion of the target RNA, specifically the segment of the target RNA which is to be labeled (see, e.g., FIG. 1B and FIG. 7A). In particular embodiments, the segment of the target RNA to be labeled is internal, i.e., not located at the most 5′ or 3′ nucleotide of the target RNA. The selection of the location for attachment of the tagging RNA to the target RNA is determined by the skilled artisan.

Methods such as site-directed mutation and solid-phase synthesis are routinely practiced in the art and can be used to change one or more nucleotides of the deoxyribozyme such that it is complementary with the desired substrates (i.e., the target RNA and tagging RNA). As used herein, the term “complementary”, when used in reference to nucleic acids (i.e., a sequence of nucleotides such as a deoxyribozyme, tagging RNA or a target RNA), refers to sequences that are related by base-pairing rules. For natural bases, the base pairing rules are those developed by Watson and Crick. As an example, for the sequence “5′-T-G-A-3′”, the complementary sequence is “5′-T-C-A-3′.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands affects the efficiency and strength of hybridization between the nucleic acid strands.

In accordance with embodiments drawn to the use of a tagging RNA which is an oligonucleotide, ligation of the tagging RNA to a target RNA can add extraneous nucleotides to the target RNA (see, e.g., FIG. 1B). Accordingly, particular embodiments embrace contacting the target RNA with a second deoxyribozyme to remove one or more tagging RNA nucleotides. Deoxyribozymes of use in accordance with this embodiment are desirably complementary with the tagging RNA and not the target RNA. As the skilled artisan can appreciate, any one of the deoxyribozymes disclosed herein can be employed in this embodiment of the present invention with particular embodiments embracing the use of the 10-23 deoxyribozyme.

The complementary portions of the deoxyribozyme with the tagging RNA and target RNA can be any suitable length depending upon the desired avidity. For example, increasing the length of the arms of the deoxyribozyme increases the number of Watson-Crick bonds, thus increasing the avidity. The opposite is true for decreasing the length of the arms. Decreasing the avidity of the arms facilitates the removal of substrate from the enzyme, thus allowing faster enzymatic turnover. Generally, each arm of the deoxyribozyme is independently 5-50 nucleotides in length.

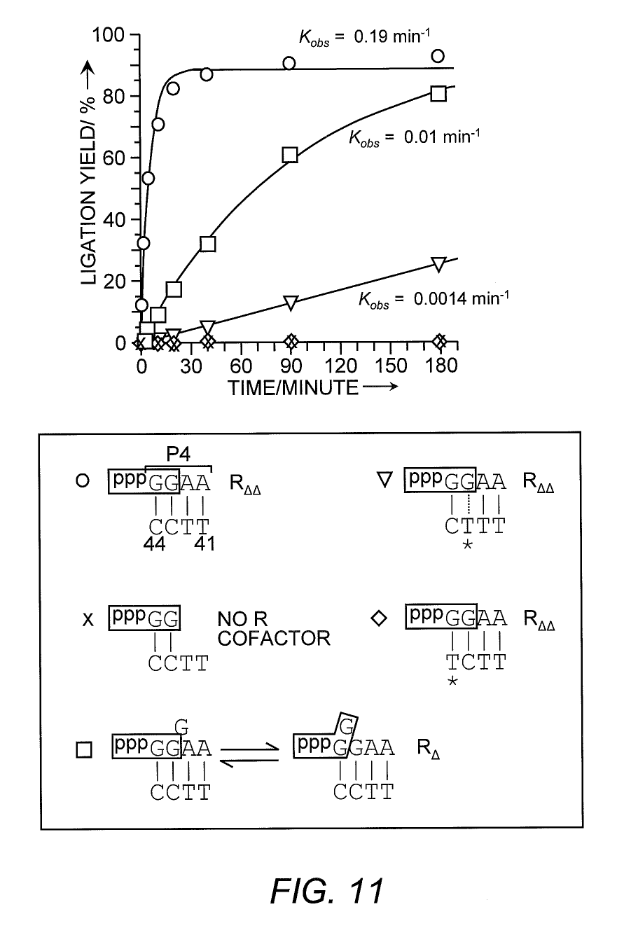

In an alternative embodiment of the present invention, the tagging RNA is a mono- or dinucleotide. As demonstrated herein (see Example 2), the 10DM24 deoxyribozyme was employed to mediate the multiple-turnover ligation reaction of a small-molecule nucleotide triphosphate (NTP) rather than a 5′-triphosphorylated oligonucleotide as an electrophilic substrate. Using the 10DM24 deoxyribozyme, attachment of GTP (i.e., pppG) and pppGpG to a target RNA molecule was achieved. Thus, the present invention embraces a method for labeling a target RNA molecule via deoxyribozyme-mediated attachment of a phosphorylated nucleotide, in particular a triphosphorylated nucleotide. In accordance with this method, the target RNA is contacted with at least one phosphorylated nucleotide in the presence of a cofactor RNA and deoxyribozyme that is complementary to the phosphorylated nucleotide (i.e., the binding site for the phosphorylated nucleotide), at least a portion of the cofactor RNA and at least a portion of the target RNA so that the phosphorylated nucleotide is site-specifically attached to the target RNA (see FIG. 7A and FIG. 11). In particular embodiments, the phosphorylated nucleotide is a mononucleotide or dinucleotide, and is desirably not more than three nucleotides in length.

In accordance with this method, the deoxyribozyme joins the target RNA with the phosphorylated nucleotide electrophile, resulting in a single nucleotide (or dinucleotide) that is site-specifically attached to the target RNA. In some embodiments, the phosphorylated nucleotide is coupled with a label prior to attachment to the target RNA. In other embodiments, the phosphorylated nucleotide is coupled with a label after attachment to the target RNA. For example, a triphosphorylated nucleotide electrophile provides a free 2′,3′-diol which is functionally equivalent to a 3′-terminus. Using conventional methods (see, e.g., Odom, et al. (1980) Biochemistry 19:5947-5954) the free 2′,3′-diol of the nucleotide can be modified by oxidation and reductive amination for subsequent coupling with a wide range of biophysical labels. Such modification of the attached nucleotide is in a fashion directly analogous to that of a true 3′-terminus.

The phosphorylated nucleotide is an electrophile and can be any phosphorylated nucleotide so long as it is compatible with the selected deoxyribozyme (e.g., the nucleotide contains a complete ribose ring). In this regard, natural nucleotides (e.g., ATP, GTP, UTP, etc.) as well as non-natural nucleotide analogs (e.g., ITP, ara-ATP, DTP, etc.) can be employed. The selection of the phosphorylated nucleotide will be dependent on the deoxyribozyme employed and can be routinely determined by one of skill in the art. Exemplary deoxyribozymes are disclosed herein, e.g., in FIG. 6 and Table 1. By way of illustration, a guanosine 5′-triphosphate (GTP) is suitably used in combination with the deoxyribozyme 10DM24. Similarly, any cofactor RNA can be employed as long as it is compatible with the selected deoxyribozyme (see, e.g., the ΔΔR and ΔR cofactors used in combination with GTP and the deoxyribozyme 10DM24) and has portions which are complementary with the deoxyribozyme. The cofactor RNA can be 1-50 nucleotides in length, and in some embodiments is 5 to 20 nucleotides in length.

Advantageously, the methods disclosed herein can be employed to test many different labels at a single site in a target RNA molecule by using a single deoxyribozyme and varying the label on the tagging RNA. Testing a particular label at different target sites simply requires the same tagging RNA and a deoxyribozyme with a binding arm that corresponds to each new target site. Because the target RNA itself has no sequence modifications, many sites can be tested with a single target sequence. This is particularly important for large RNA targets, for which preparation of mutants is relatively cumbersome. By varying the location of the functionalized nucleotide in the tagging RNA, the DECAL approach permits adjusting the distance of the label from the target RNA, which may be important for various applications.

Labeled RNA molecules have a wide variety of uses, which encompass essentially any context in which separation, isolation, purification, detection or identification of an RNA is desired and/or in which alteration of a characteristic(s) of the RNA is desired. For example, a specific RNA molecule can be labeled in a cell lysate and subsequently detected by a detection technique (e.g., by colorimetric, fluorescence, electrophoretic, electrochemical, spectroscopic, chromatographic, densitometric, or radiographic techniques) to indicate the presence or concentration of the target RNA. The presence of a reporter molecule will typically be determined by the detection technique (e.g., fluorophore reporters for fluorescent techniques and radiolabels for radiographic techniques.)

The invention is described in greater detail by the following non-limiting examples.

### EXAMPLE 1

### Incorporation of Tagging RNA into P4-P6 RNA

Materials and Methods. DNA oligonucleotides were prepared at IDT (Coralville, Iowa). Short target RNA substrates for comprehensive sequence-dependence studies and the aminoallyl-modified tagging RNA were prepared by in vitro transcription with T7 RNA polymerase and a synthetic double-stranded DNA template that was prepared by annealing two DNA oligonucleotides (Milligan, et al. (1987) Nucleic Acids Res. 15:8783-8798). The P4-P6 RNA and its mutant forms were prepared by in vitro transcription with T7 RNA polymerase and a linearized plasmid template (Silverman and Cech (1999) Biochemistry 38:8691-8702; Silverman and Cech (1999) Biochemistry 38:14224-14237). DNA and RNA oligonucleotides and transcripts were purified by denaturing PAGE as described previously (Flynn-Charlebois, et al. (2003) J. Am. Chem. Soc. 125:2444-2454; Wang and Silverman (2003) Biochemistry 42:15252-15263).

Design and Synthesis of Tagging RNA. The sequence of the tagging RNA was designed on the basis of several considerations. Desirably the biophysical label was to be relatively close to the target RNA. Therefore, the 5-aminoallylcytidine nucleotide used for attaching the label to the tagging RNA was placed near the 5′-terminus of the transcript. Because T7 RNA polymerase requires G or A as the initiating nucleotide (Milligan, et al. (1987) supra; Coleman, et al. (2004) Nucleic Acids Res. 32:e14), the closest possible position for the aminoallyl-nucleotide (which is commercially available as the 5′-triphosphate of C or U) is the second position from the 5′-terminus. To ensure that the tagging RNA contained only a single label, the aminoallyl-nucleotide must be incorporated only once into the transcript. On the basis of these considerations and to avoid potential hybridization with any portion of the P4-P6 RNA sequence, the sequence of the unlabeled tagging RNA was 5′-GC^{aa}A AGA GAU GGU GAU GGG A-3′ (SEQ ID NO:15), where C^{aa} denotes 5-aminoallyl-C. 5-Aminoallyl-CTP was used instead of the UTP derivative because of higher transcription yield. The two DNA template oligonucleotides were 5′-TCC CAT CAC CAT CTC TTG CTA TAG TGA GTC GTA TTA CAG CGT GCG T-3′ (SEQ ID NO:16) and 5′-ACG CAC GCT GTA ATA CGA CTC ACT ATA-3′ (SEQ ID NO:17), wherein the coding sequence is underlined. Transcription conditions were as follows: 1 μM each DNA template, 40 mM Tris (pH 8.0), 30 mM MgCl₂, 10 mM DTT, 2 mM spermidine, 4 mM each ATP, GTP, and UTP, and 2 mM 5-aminoallyl-CTP (TriLink BioTechnologies, San Diego, Calif.). After incubation of the 200-800-μL sample at 37° C. for 5 hours, the transcript was purified by 20% denaturing PAGE. Typical yields after extraction and ethanol precipitation were 1.4-3.0 nmol of aminoallyl-modified RNA transcript per 100 μL of transcription reaction.

Coupling the Label to the Tagging RNA. The aminoallyl-modified tagging RNA transcript was coupled with the amine-reactive NHS ester of biotin (ChemGenes, Wilmington, Mass.), DABCYL (AnaSpec, San Jose, Calif.), 5(6)-fluorescein (Pierce Biotechnology, Rockford, Ill.) or 5(6)-TAMRA (Molecular Probes, Eugene, Oreg.). For biotin and TAMRA, 5 μM aminoallyl-RNA and 5 mM NHS ester were incubated with 0.2 mM EDTA in 100 mM sodium phosphate (pH 8.0) and 50% (v/v) DMSO at 37° C. for 24 hours (biotin) or 3 hours (TAMRA). For DABCYL, 5 μM aminoallyl-RNA and 100 mM NHS ester were incubated with 0.2 mM EDTA in 100 mM sodium phosphate (pH 8.0) and 50% (v/v) DMSO at 37° C. for 24 hours. For fluorescein, 10 μM aminoallyl-RNA and 21 mM NHS ester were incubated with 0.2 mM EDTA in 70 mM sodium bicarbonate (pH 9.0) and 30% (v/v) DMSO at 37° C. for 3 hours. Unreacted labeling reagent was removed by ethanol precipitation, and labeled transcripts were separated from unlabeled transcripts by 20% denaturing PAGE. Labeling reactions were performed on the 1 nmol scale. Isolated yields of labeled transcripts after gel extraction and ethanol precipitation were ˜27% for biotin, 5-10% for DABCYL, ˜20% for fluorescein, and ˜15% for TAMRA.

Assays of Labeling Generality Using 10DM24. The ability of the 10DM24 deoxyribozyme (Zelin, et al. (2006) supra) to utilize the various tagging RNA transcripts was assayed with a systematic series of short target RNAs. In accordance with established nomenclature (Flynn-Charlebois, et al. (2003) supra), the target RNA serves as the left-hand substrate and is designated L, whereas the tagging RNA is the right-hand substrate and is designated R (FIG. 1B). The initial target RNA (parent sequence 5′-GGA UAA UAC GAC UCA CUA UA-3′ (SEQ ID NO:18) with branch-site adenosine underlined) was the L substrate originally used in the selection that led to identification of 10DM24 (Zelin, et al. (2006) supra). Target L substrates that have systematic sequence changes relative to the parent sequence were tested (Table 2).

TABLE 2

| | | SEQ ID |
|---|---|---|
| L substrate | Sequence (5′->3′) | NO: |
| Parent | GGA UAA UAC GAC UCA CUA UA | 18 |
| Transitions | GGA CGG CGU AAU CUG UUA UA | 19 |
| Transversions-1 | GGA GCC GCA UAA GAC AUA UA | 20 |
| Transversions-2 | GGA AUU AUG CAG AGU GUA UA | 21 |

In addition to the branch-site A, the 5′-GGA (included for efficient transcription) and the four nucleotides at the 3′-terminus were left unchanged. The sequence changes were denoted as transitions (A [symbol] G, U [symbol] C), transversions-1 (A [symbol] C, G [symbol] U), and transversions-2 (A [symbol] U, G [symbol] C). The corresponding DNA changes were made at each Watson-Crick base-paired position of 10DM24. Each L substrate was tested with a series of tagging RNA transcripts. The tagging RNA was either entirely unmodified, unlabeled (i.e., 5-aminoallyl-C at the second nucleotide position), or labeled at the aminoallyl group with biotin, DABCYL, fluorescein, or TAMRA as described above. All assays with 10DM24 were performed accordingly to established methods (Flynn-Charlebois, et al. (2003) supra), in which the 5′-³²P-radiolabeled L substrate was the limiting reagent relative to 10DM24 (E) and the tagging RNA (R). The ratio L:E:R was 1:3:6, with E equal to 0.3 μM. Reactions were performed in 50 mM CHES (pH 9.0), 150 mM NaCl, 2 mM KCl, and 40 mM MgCl₂ at 37° C. for up to 2 hours. At appropriate time points, 1.5 μL was removed from the sample and quenched into 8 μL stop solution (80% formamide, 1× TB (89 mM each Tris and boric acid, pH 8.3), and 50 mM EDTA, containing 0.025% bromophenol blue and xylene cyanol). Samples were separated by 20% denaturing PAGE and imaged with a PHOSPHORIMAGER. The resulting data were fit to yield=Y·(1-e^{-kt}), where k=k_{obs} and Y=final yield.

The 10DM24 deoxyribozyme successfully used the various tagging RNA substrates in many but not all target sequence contexts (FIGS. 1C-H). For all tagging RNAs, the parent L sequence had the highest ligation yield; L with either transversions-1 or transversions-2 as the sequence changes was slower but still generally high-yielding. In contrast, L with transitions as the sequence changes was a poorer target.

Assays of Using P4-P6 as Target RNA. As another test of deoxyribozyme-catalyzed labeling of RNA, 10DM24 was assayed for the ability to label nucleotides in the 160-nt Tetrahymena group I intron P4-P6 domain (Murphy and Cech (1993) Biochemistry 32:5291-5300; Murphy and Cech (1994) J. Mol. Biol. 236:49-63; Smalley and Silverman (2006) Nucleic Acids Res. 34:152-166; Cate, et al. (1996) Science 273:1678-1685). The labeling sites in P4-P6 were chosen on the basis of the X-ray crystal structure (Cate, et al. (1996) supra). Only nucleotides with accessible 2′-hydroxyl groups were chosen, and choices were restricted to adenosines on the basis of the 10DM24 branch-site preference (Zelin, et al. (2006) supra). Approximately 24 adenosines were identified as accessible. The ten tested adenosines were scattered throughout P4-P6, including within the central region of the RNA (where modifications that require subsequent ligations are challenging). There were no obvious similarities among the RNA sequences surrounding the adenosines.

The labeling assays were performed as described above. Relative to L, 100 equivalents of a disruptor (D) DNA oligonucleotide that interferes with the local RNA secondary structure were added, thereby allowing 10DM24 to bind nucleotides within P4-P6 flanking the target site. Samples were annealed in 7 μL of 5 mM HEPES (pH 7.5), 15 mM NaCl, and 0.1 mM EDTA by heating at 95° C. for 3 minutes and cooling on ice for 5 minutes. The reaction buffer was added and the samples were incubated at 3720 C. for 2 minutes, then MgCl₂ was added. The final incubation conditions were 50 mM CHES (pH 9.0), 150 mM NaCl, 2 mM KCl, and 40 mM MgCl₂ in a volume of 10 μL. Reactions were incubated at 37° C. for 2 hours and quenched with stop solution. The products were analyzed by 6% denaturing PAGE.

The data are shown in FIG. 2B. Eight of the ten tested nucleotide locations were readily derivatized (>50%) with the entirely unmodified R transcript, which contained cytidine instead of 5-aminoallyl-C at the second position. Six nucleotide locations were readily derivatized using R that had a 5-aminoallyl-C at the second position. Although inclusion of the tested biophysical labels (biotin, fluorescein, or TAMRA) within the tagging RNA generally led to a decrease in yield, multiple sites were successfully labeled in preparatively useful yield (>40%) with biotin (six sites), fluorescein (five sites including A231) and TAMRA (one site, A146).

Preparative Double-Labeling of P4-P6. Preparative double-labeling of P4-P6 with fluorescein and TAMRA was achieved in two steps. The first tag (either with or without attached fluorescein) was attached at nucleotide A231. After PAGE purification, the second tag (with or without attached TAMPA) was attached at nucleotide A146. The RNAs with one or zero chromophores were synthesized as controls and to facilitate FRET analysis.

For ligation at A231, the ratio R:E:L:D was 1.0:1.1:1.2:2.0, where R was equal to 12.5-25 μM. Samples were annealed in 28 μL of 5 mM HEPES (pH 7.5), 15 mM NaCl, and 0.1 mM EDTA by heating at 95° C. for 3 minutes and cooling on ice for 5 minutes. The reaction buffer was added and the samples were incubated at 37° C. for 2 minutes, then MgCl₂ was added. The final conditions were 50 mM CHES (pH 9.0), 150 mM NaCl, 2 mM KCl, and 40 mM MgCl₂ in a volume of 40 μL. Reactions were incubated at 37° C. for 2 hours and quenched with 40 μL stop solution. The A231-modified RNAs were separated from unmodified P4-P6 by 6% denaturing PAGE.

For ligation at A146, the ratio L:E:R:D was 1.0:1.1:1.2:2.0, where L was equal to ˜1-3.4 μM. Reactions were performed under the same reaction conditions as used for the ligation at A231. The doubly modified P4-P6 RNA was readily separated from the singly modified P4-P6 RNA by 6% denaturing PAGE.

Nondenaturing Gel Electrophoresis. The native PAGE experiments were performed at 35° C. according to established methods (Silverman and Cech (1999) supra; Young and Silverman (2002) Biochemistry 41:12271-12276; Miduturu and Silverman (2005) J. Am. Chem. Soc. 127:10144-10145; Purtha, et al. (2005) J. Am. Chem. Soc. 127:13124-13125), except each RNA sample included 10 μmol of U₁₃ carrier RNA (Miduturu and Silverman (2005) supra). The titration curves for unmodified P4-P6 and for doubly tagged P4-P6 (A231-fluorescein and A146-TAMRA) are shown in FIG. 3. From these data, [Mg²⁺]_{1/2} is 0.72 mM for unmodified P4-P6 and 0.88 mM for doubly tagged P4-P6 (ΔΔG°=0.5 kcal/mol).

Tagging RNA Truncation by a 10-23 Deoxyribozyme. Ligation of a single tagging RNA to the target RNA adds 19 nucleotides to the target. Although appending these single-stranded nucleotides is not anticipated to affect the folding of a large RNA target (Miduturu and Silverman (2005) supra), as demonstrated directly by native PAGE for P4-P6 (FIG. 3), the additional nucleotides could be problematic on certain RNA targets. Therefore, a method was developed for removing some of the added nucleotides using the 10-23 deoxyribozyme (Santoro and Joyce (1997) Proc. Natl. Acad. Sci. USA 94:4262-4266), leaving only eight nucleotides of each truncated tagging RNA (FIG. 4).

The truncation reactions were performed with P4-P6 doubly tagged at A231 with fluorescein and at A146 with TAMRA, along with an excess of the 10-23 deoxyribozyme. The ratio of doubly tagged P4-P6 to 10-23 deoxyribozyme was 1:6 (0.1 and 0.6 μM) or 1:600 (0.03 and 18 μM). 5′-³²P-Radiolabeled doubly tagged P4-P6 was included in a trace amount in each sample, with the remainder of the RNA as 5′-unradiolabeled (no disruptor oligonucleotides were included). Samples were annealed in 5 mM HEPES (pH 7.5), 15 mM NaCl and 0.1 mM EDTA by heating at 95° C. for 3 minutes and cooling on ice for 5 minutes. The reaction buffer was added and the samples were incubated at 37° C. for 2 minutes, then MgCl₂ or MnCl₂ was added. The final incubation conditions were 50 mM HEPES (pH 7.5), 150 mM NaCl, and either 10 mM MgCl₂, 40 mM MgCl₂, 5 mM MnCl₂, or 20 mM MnCl₂ at 37° C. in a volume of 10 μL. At appropriate timepoints, 1.5 μL was removed from the sample and quenched into 8 μL stop solution. Samples were separated by 6% denaturing PAGE and imaged with a PHOSPHORIMAGER.

The results of this analysis indicated that the 10-23 deoxyribozyme readily truncated both tagging RNAs when they were attached to P4-P6. The presence of fluorescein and TAMRA on the tags does not inhibit truncation. Increasing the excess of 10-23 from 6-fold to 600-fold had little effect, and both Mg²⁺ and Mn²⁺ were effective. This successful truncation indicates that the tagging strands are freely accessible to the 10-23 deoxyribozyme.

Steady-State FRET Experiments. The Mg²⁺-dependent folding of doubly tagged P4-P6 was analyzed by steady-state FRET using a Thermo AB2 spectrometer. The sample temperature was maintained at 25° C. with a recirculating water bath. For all scans, the excitation and emission bandpass settings were 4 nm and 8 nm with a resolution of 1 nm. Three different versions of the P4-P6 sequence were doubly tagged for FRET studies: wild-type P4-P6 (P4-P6-wt), non-foldable P4-P6 (P4-P6-bp), and a P4-P6 mutant with two adenosine nucleotides inserted within the GAAA tetraloop (Young and Silverman (2002) supra). The latter mutant was previously shown by native PAGE to have a significantly higher [Mg²⁺]_{1/2} value (by approximately ten-fold) than wild-type P4-P6 (Young and Silverman (2002) supra). Each doubly tagged P4-P6 variant had fluorescein at A231 and TAMRA at A146. Donor-only control samples were also prepared with fluorescein at A231 and an unlabeled aminoallyl tag at A146.

For each titration, the initial sample was 14 nM RNA in 1× TB buffer in a volume of 70 μL. During the titration, aliquots of MgCl₂ in 1× TB were added to the sample, which was mixed manually in the cuvette by pipetting and re-equilibrated at 25° C. prior to starting the scan. The titrations were performed from 0 to 200 mM Mg²⁺. For measurements of donor (fluorescein) fluorescence in the presence and absence of acceptor and measurements of acceptor (TAMRA) fluorescence due to FRET, samples were excited at 494 nm and the emission spectra were obtained from 505-650 nm. For measurements of acceptor fluorescence due to direct excitation, samples were excited at 565 nm and the emission spectra were obtained over the range 575-650 nm. The scan rate was set at 4 nm/s to minimize fluorescein photobleaching, which was estimated to be <2% during the course of a complete FRET experiment. All spectra were corrected for dilution due to MgCl₂ addition and for buffer background fluorescence.

The FRET efficiency (E_{FRET}) was determined by the (ratio)_{A} method (Clegg (2000) supra). The spectrum of the donor-only P4-P6 was normalized to the donor emission peak (521 nm) of the doubly tagged P4-P6 spectrum. The normalized donor-only spectrum was then subtracted from the doubly tagged P4-P6 spectrum, providing the extracted acceptor spectrum. The extracted acceptor spectrum, which is a measure of the fluorescence (F) of the acceptor from excitation at ν′=494 nm via both direct excitation and energy transfer with emission at ν₁=575-650 nm, was then divided by the acceptor spectrum from excitation at ν″=565 nm with emission at ν₂=575-650 nm to give (ratio)_{A} as follows:

$${( ratio )}_{A} = \frac{F ( {v}_{1} , {v}^{′} )}{F ( {v}_{2} , {v}^{″} )}$$

E_{FRET} was then calculated from (ratio)_{A} as follows:

$${( ratio )}_{A} = \{ {E}_{FRET} {d}^{+} [ \frac{{ɛ}^{D} ( {v}^{′} )}{{ɛ}^{A} ( {v}^{″} )} ] + \frac{{ɛ}^{A} ( {v}^{′} )}{{ɛ}^{A} ( {v}^{″} )} \} \frac{{Φ}^{A} ( {v}_{1} )}{{Φ}^{A} ( {v}_{2} )}$$

Because the samples were 100% labeled with donor, d⁺=1. Because ν₁=ν₂, the final term in the equation was also equal to 1. ε^{D}(ν′)/ε^{A}(ν″) was calculated using extinction coefficients of 83,000 cm⁻¹ M⁻¹ for fluorescein and 91,000 M⁻¹ cm⁻¹ for TAMRA (values according to IDT). ε^{A}(ν′)/ε^{A}(ν″) was determined from the excitation spectrum of P4-P6 labeled with TAMRA at A146 and an aminoallyl tag at A231 (i.e., acceptor-only sample). The reported E_{FRET} values were the average of two Mg²⁺ titrations. The data were fit in a similar fashion as the native PAGE data by using the equation (E_{FRET})_{obs}=((E_{FRET})_{low}+(E_{FRET})_{high}·K·[Mg²⁺]ⁿ)/(1+K·[Mg²⁺]ⁿ), where (E_{FRET})_{obs} is the observed E_{FRET} as a function of [Mg²⁺]; (E_{FRET})_{low} and (E_{FRET})_{high} are the limiting low and high values of E_{FRET}; and K, n and [Mg²⁺]_{1/2} are defined as known in the art (Silverman and Cech (1999) Biochemistry 38:8691-8702). From the curve fits in FIG. 5, the [Mg²⁺]_{1/2} values for P4-P6-wt, P4-P6 tetraloop mutant, and P4-P6-bp were 1.57 mM, 5.5 mM, and 33 mM, respectively.

### EXAMPLE 2

### Mononucleotide Incorporation into Target RNA

Materials. The dNTPs were from Fermentas (Hanover, Md.); ITP and G^{ox}TP (i.e., periodate-oxidized GTP) were from Sigma (St. Louis, Mo.); and ddGTP, ddATP, DTP, d2AP-TP, and ara-ATP were from Trilink Biotechnologies (San Diego, Calif.). DNA oligonucleotides and RNA oligonucleotides with 5′-pyrimidine nucleotides were prepared at IDT (Coralville, Iowa). RNA oligonucleotides with 5′-purine nucleotides were prepared by in vitro transcription with T7 RNA polymerase and a synthetic DNA template. The standard T7 promoter sequence (5′-ACG CAC GCT GTA ATA CGA CTC ACT ATA-3′, SEQ ID NO:17)(Milligan, et al. (1987) supra) was used for transcriptions that were initiated with GTP. For transcriptions that were initiated with ATP, an alternative promoter sequence was used in which the 3′-terminal nucleotide was changed from A to T (5′-ACG CAC GCT GTA ATA CGA CTC ACT ATT-3′, SEQ ID NO:22; Coleman, et al. (2004) supra; Huang, et al. (2003) RNA 9:1562-1570). As a consequence, the corresponding nucleotide in the reverse DNA oligonucleotide was A instead of T, to retain Watson-Crick base-pairing. Transcription reactions with T7 RNA polymerase were performed using 1 μM reverse strand and 1 μM promoter strand in 40 mM Tris-HCl, pH 8.0, 30 mM MgCl₂, 10 mM DTT, 4 mM each NTP, and 2 mM spermidine at 37° C. for 3-5 hours. All DNA and RNA oligonucleotides were purified by denaturing PAGE with running buffer 1× TBE (89 mM each Tris and boric acid and 2 mM EDTA, pH 8.3) according to known methods (Flynn-Charlebois, et al. (2003) supra; Wang and Silverman (2003) supra).

DNA Oligonucleotides. The 10DM24 deoxyribozyme and its variants are listed in TABLE 3. The P4 region is in bold and italics (i.e., nucleotides 41-44). The bold nucleotides represent the catalytic loop regions, and the non-bold nucleotides constitute the P1, P2, and P3 binding regions for RNA substrates and/or cofactors.

TABLE 3

| | | SEQ |
|---|---|---|
| | | ID |
| 10DM24 | Sequence | NO: |
| Original | CCGTCGCCATCTCCCGTAGGTGAAGGGCGTGAGGGTTCCA [symbol] CGTATTATCC | 12 |
| C44T | CCGTCGCCATCTCCCGTAGGTGAAGGGCGTGAGGGTTCCA [symbol] CGTATTATCC | 23 |
| C44A | CCGTCGCCATCTCCCGTAGGTGAAGGGCGTGAGGGTTCCA [symbol] CGTATTATCC | 24 |
| C44G | CCGTCGCCATCTCCCGTAGGTGAAGGGCGTGAGGGTTCCA [symbol] CGTATTATCC | 25 |
| C43T | CCGTCGCCATCTCCCGTAGGTGAAGGGCGTGAGGGTTCCA [symbol] CGTATTATCC | 26 |
| C43A | CCGTCGCCATCTCCCGTAGGTGAAGGGCGTGAGGGTTCCA [symbol] CGTATTATCC | 27 |
| C43G | CCGTCGCCATCTCCCGTAGGTGAAGGGCGTGAGGGTTCCA [symbol] CGTATTATCC | 28 |
| C43G/C44T | CCGTCGCCATCTCCCGTAGGTGAAGGGCGTGAGGGTTCCA [symbol] CGTATTATCC | 29 |
| C43A/C44T | CCGTCGCCATCTCCCGTAGGTGAAGGGCGTGAGGGTTCCA [symbol] CGTATTATCC | 30 |
| C43T/C44T | CCGTCGCCATCTCCCGTAGGTGAAGGGCGTGAGGGTTCCA [symbol] CGTATTATCC | 31 |

RNA Oligonucleotides. The RNA substrate (L) was 5′-GGA UAA UAC GAC UCA C-3′ (SEQ ID NO:13), wherein with branch-site adenosine is underlined. RNA substrates (R, i.e., target RNAs) with mutations in P4 region are listed in Table 4. The P4 region is in bold and mutations are underlined.

TABLE 4

| | | |
|---|---|---|
| Substrate | Sequence | SEQ ID NO: |

| | | |
|---|---|---|
| R | GGAAGAGAUGGCGACGG | 14 |
| R-G1A | AGAAGAGAUGGCGACGG | 32 |
| R-G1U | UGAAGAGAUGGCGACGG | 33 |
| R-G1C | CGAAGAGAUGGCGACGG | 34 |
| R-G2A | GAAAGAGAUGGCGACGG | 35 |
| R-G2U | GUAAGAGAUGGCGACGG | 36 |
| R-G2C | GCAAGAGAUGGCGACGG | 37 |

Truncated cofactor RNAs (RΔ or RΔΔ) of the tagging RNA are listed in Table 5.

TABLE 5

| | | |
|---|---|---|
| Cofactor RNA | Sequence | SEQ ID NO: |

| | | |
|---|---|---|
| RΔ | GAAGAGAUGGCGACGG | 38 |
| RΔ-G2A | AAAGAGAUGGCGACGG | 39 |
| RΔ-G2U | UAAGAGAUGGCGACGG | 40 |
| RΔ-G2C | CAAGAGAUGGCGACGG | 41 |
| RΔΔ | AAGAGAUGGCGACGG | 42 |

General Description of Kinetic Assays. All kinetic assays with the 10DM24 deoxyribozyme and its variants were performed according to established methods (Flynn-Charlebois, et al. (2003) supra; Wang and Silverman (2003) supra). The 5′-³²P-labeled RNA substrate (L for “left-hand substrate”) that provides the branch-site adenosine was the limiting reagent relative to 10DM24 (E) and the truncated cofactor RNA (RΔ; R originally derived from “right-hand substrate”). The ratio L:E:RΔ was 1:10:30, with 0.25 μM 10DM24. The cofactor RΔ was 5′-phosphorylated unless otherwise stated. The RNA substrate L, deoxyribozyme 10DM24 and cofactor RΔ were annealed in 5 mM HEPES, pH 7.5, 15 mM NaCl, 0.1 mM EDTA by heating at 95° C. for 3 minutes and cooling on ice for 5 minutes. The ligation reactions were performed with the appropriate concentration of NTP substrate (0.05-50 mM) at a final buffer concentration of 100 mM CHES, pH 9.0, 150 mM NaCl, 2 mM KCl, and 40-400 mM MgCl₂ at 37° C. for up to 5 hours. The combination of 1 mM NTP and 40 mM MgCl₂ was defined as “standard incubation conditions”; 10 mM NTP and 150 mM MgCl₂ was defined as “enhanced incubation conditions”. At appropriate timepoints, aliquots were removed from the sample, quenched into stop solution (80% formamide, 1× TB [89 mM each Tris and boric acid, pH 8.3], and 50 mM EDTA containing 0.025% bromophenol blue and xylene cyanol) and stored at −20° C. prior to analysis. Samples were separated by 20% denaturing PAGE at 30 W for 115 minutes and imaged with a PHOSPHORIMAGER. The resulting data were fit to the equation yield=Y·(1−e^{-kt}), where k=k_{obs} and Y=final yield.

Synthesis of C2-C3-cleaved GTP (G^{clv}-TP). The acyclic GTP analogue G^{clv}TP was prepared from commercially available guanosine 5′-triphosphate 2′,3′-dialdehyde (periodate-oxidized GTP or G^{clv}TP, Sigma, 85-90%). A sample of G^{ox}TP (2.6 mg, 5 μmol) was dissolved in H₂O (80 μL, final concentration 50 mM) and combined with sodium borate buffer, pH 8.0 (10 μL of 1 M, final concentration 100 mM) and sodium borohydride (10 μL of 1 M solution in H₂O, prepared immediately before use with H₂O at 4° C.; Hawley, et al. (1978) Biochemistry 17:2082-2086). The reaction solution, from which instantaneous gas evolution was observed, was incubated on ice for 30 minutes (Scheme 1).

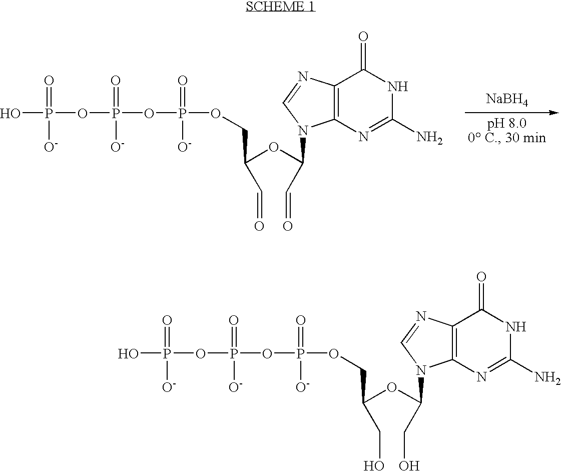

The product was precipitated by the addition of acetone (1 mL). The sample was kept on dry ice for 30 minutes, and the precipitate was recovered by centrifugation at 16000 g and 4° C. for 30 minutes. The pellet was dissolved in H₂O (30 μ) and the pH was adjusted to 7.5 by the addition of 100 mM HCl (ca. 10 μL). The sodium salt of the crude G^{clv}TP was again precipitated by the addition of acetone. After cooling on dry ice and centrifugation, the pellet was dried under vacuum then dissolved in 450 μL D₂O, and a ³¹P NMR spectrum was recorded. The crude sample contained ˜30% of the C2-C3-cleaved guanosine diphosphate derivative (G^{clv}DP; the diphosphate impurity was present in the starting material). A portion of the crude G^{clv}TP sample was purified by RP-HPLC. The product-containing fractions were combined and evaporated to dryness. To remove excess TEAA, the product was dissolved in 250 μL H₂O and evaporated four times. Finally, the product was dissolved in H₂O, and the concentration was determined by UV absorbance (ε₂₆₀ 11700 L·mol⁻¹·cm⁻¹). G^{clv}TP: ESI-MS calcd. for C₁₀H₁₈N₅O₁₄P₃ [M−H]⁻ 524.2, found [M−H]⁻ 524.1. ³¹P NMR (162 MHz, D₂O) δ −5.0 (d, J=20 Hz, Pγ), −10.0 (d, J=20 Hz, Pα), −21.0 (t, J=20 Hz, Pβ) ppm. G^{clv}DP: ESI-MS calcd. for C₁₀H₁₆N₅O₁₁P₂ [M−H]⁻ 444.2, found [M−H]⁻ 444.1. ³¹P NMR (162 MHz, D₂O) δ −5.4 (d, J=23 Hz, Pβ), −9.7 (d, J=23 Hz, Pα). Assignment of phosphorus resonances was based on ³¹P NMR for GTP (Solomon, et al. (2001) Org. Lett. 3:4311-4314).

Synthesis of Acyclovir Triphosphate (G^{acv}TP). The synthesis of G^{acv}TP from guanine is depicted in Scheme 2.

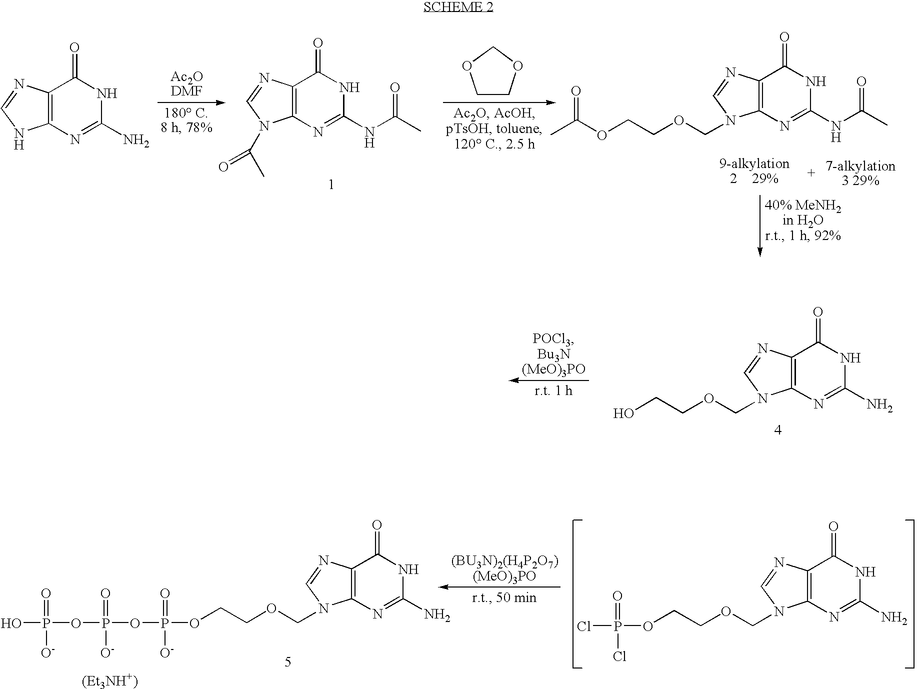

N²,9-Diacetylguanine (1) was prepared according to established methods (Zou & Robins (1987) Can. J. Chem. 65:1436-1437) by heating a suspension of guanine (4.0 g, 26 mmol) in dry DMF (30 mL) and acetic anhydride (15 mL) at 180° C. for 8 hours. A clear, brown solution was obtained, from which the off-white product crystallized upon cooling to room temperature. The product was filtered, washed with ethanol, and dried under vacuum. Yield: 4.8 g 1 (78%). ¹H NMR (400 MHz, DMSO-d₆) δ 2.21, 2.81 (2 s, 6H, 2 CH₃CONH), 8.46 (s, 1H, H—C(8)), 11.78, 12.24 (2 s, 2H, 2 NH) ppm. ¹³C NMR (100 MHz, DMSO-d₆) δ 23.9, 24.7 (2 CH₃CONH), 121.5, 137.5, 148.4, 154.6, 168.0, 173.8 (2 CONH) ppm.

N²-Acetyl-9-(2-acetoxyethoxymethyl)guanine (2) was prepared according to known methods (Gao & Mitra (2001) Synth. Commun. 31:1399-1419). A mixture of acetic acid (0.26 mL, 4.5 mmol, 1.3 equiv.) and acetic anhydride (1.5 mL, 16.2 mmol, 4.8 equiv.) was cooled to 0° C. in an ice-water bath, and pTsOH (112 mg, 0.6 mmol, 0.2 equiv.) was added. 1,3-Dioxolane (1.2 mL, 17.4 mmol, 5.1 equiv.) was added drop-wise to the stirred solution under continued cooling in the ice-water bath. A suspension of N²,9-diacetylguanine 1 (800 mg, 3.4 mmol) in toluene (5 mL) was added. The solution was allowed to warm to room temperature and was then heated at reflux for 2.5 hours (120° C. oil bath temperature). TLC (9:1 dichloromethane/methanol) showed complete consumption of 1 and the formation of two products in ˜1:1 ratio. The biphasic mixture (oily brown phase underneath colorless phase) was cooled to room temperature and the solvent was evaporated. The oily residue was separated by column chromatography on SiO₂ with 2-10% methanol in dichloromethane (1% steps, 100 mL each). Both products were isolated and characterized as the 9-alkylation (2) and 7-alkylation (3) products by the characteristic chemical shift differences of H—C(8) and NCH₂O in the 1H NMR spectrum (Gao & Mitra (2001) supra). The undesired 7-alkyation product 3 is less polar and was recovered from early fractions (#1-16), and the desired N²-acetyl-9-(2-acetoxyethoxymethyl)guanine 2 was recovered from late fractions (#19-55). Yield: 300 mg 2 (29%). ¹H NMR (400 MHz, DMSO-d₆) δ 1.93, 2.16 (2 s, 6H, 2 CH₃CONH), 3.66, 4.05 (2 m, 4H, OCH₂CH₂OCO), 5.46 (s, 2H, NCH₂O), 8.13 (s, 1H, H—C(8)), 11.79, 12.06 (2 s, 2H, 2 NH) ppm. Yield: 300 mg 3 (29%). ¹H NMR (400 MHz, DMSO-d₆) δ 1.93, 2.15 (2 s, 6H, 2 CH₃CONH), 3.69, 4.04 (2 m, 4H, OCH₂CH₂OCO), 5.66 (s, 2H, NCH₂O), 8.36 (s, 1H, H—C(8)), 11.63, 12.16 (2 s, 2H, 2 NH) ppm.

9-(2-Hydroxyethoxymethyl)guanine (acyclovir; 4) was prepared by treatment of 3 (100 mg, 0.32 mmol) with 40% aqueous methylamine solution (1 mL) at room temperature for 1 hour in a tightly stoppered round bottom flask (Gao & Mitra (2001) supra). The solvent was evaporated and the residue was triturated with ethanol. The product was recovered by centrifugation (16000 g, at room temperature for 10 minutes) and dried under vacuum. Yield: 67 mg 4 (92%). ¹H NMR (400 MHz, DMSO-d₆) δ 3.44 (s, 4H, OCH₂CH₂OCO), 4.67 (s, 1H, OH), 5.32 (s, 2H, NCH₂O), 6.49 (br. s, 2H, NH₂), 7.80 (s, 1H, H—C(8)), 10.60 (br. s, 1H, NH) ppm. ESI-MS: m/z calcd. for C₈H₁₀N₅O₃ [M−H]⁻ 224.2, found [M−H]⁻ 224.1.

Acyclovir triphosphate has previously been synthesized via enzymatic and chemical routes (Reardon & Spector (1989) J. Biol. Chem. 264:7405-7411; Furman, et al. (1979) J. Virol. 32:72-77). Many chemical approaches are known for the synthesis of nucleoside analog triphosphates (Burgess & Cook (2000) Chem. Rev. 100:2047-2059). A common procedure that involves formation of a dichlorophosphate intermediate was employed. Acyclovir 4 (50 mg, 0.22 mmol) was coevaporated twice with pyridine (2×5 mL), dried under vacuum, placed under argon atmosphere, and dissolved in trimethyl phosphate (0.5 mL). Tributylamine (58 μL, 0.24 mmol, 1.1 equiv.) and POCl₃ (23 μL, 0.24 mmol, 1.1 equiv.) were added under argon and the solution was stirred at room temperature for 1 hour. Then, a solution of tributylammonium pyrophosphate (108 mg, 0.24 mmol, 1 equiv. based on POCl₃) in trimethyl phosphate (1 mL) was added, and the clear solution was stirred at room temperature for 30 minutes. For TLC monitoring (6:3:1 iPrOH/NH₄OH/H₂O), a sample was quenched into triethylamine. The precipitate was recovered by centrifugation and dissolved in 50 mM triethylammonium bicarbonate, pH 8.0 (TEAB) for spotting onto a silica gel TLC plate. Multiple products and unreacted starting material were observed. After 50 minutes, triethylamine (1.8 mL, 60 equiv.) was added to the reaction mixture. The resulting precipitate was recovered by centrifugation and dissolved in 2 mL 50 mM TEAB. The aqueous solution was allowed to stand at room temperature for 2 hours to ensure hydrolysis of the cyclic triphosphate intermediate. The crude product was loaded onto an anion-exchange column (BIOGEL A, BIO-RAD, 1×12 cm) which was previously equilibrated with 50 mM TEAB. The products were eluted with a stepwise gradient (50 mM then 100 mM steps) of 50-400 mM TEAB, and the fractions were monitored by TLC. The separation was poor, and a mixture of products was obtained. The presence of the desired G^{acv}TP was confirmed by ESI-MS. A fraction of the sample was further purified by anion exchange chromatography on DEAE-SEPHADEX A25 (0.5 g of DEAE-SEPHADEX A25 was swelled in 50 mM TEAB; manual flash column, ca. 1×3 cm). The product was eluted with a stepwise gradient (50 mM then 100 mM steps) of 50-800 mM TEAB in 1 mL fractions. The fractions were analyzed by UV absorbance at 260 nm. The product-containing fractions were combined and evaporated to dryness. To remove excess TEAB, the product was dissolved in 250 μL H₂O and evaporated four times. A stock solution of the product was prepared by dissolving the pellet in 180 μL H₂O (the final concentration was 10 mM as determined by UV absorbance, using ε₂₆₀=11700 L·mol⁻¹·cm⁻¹). The product was further analyzed by RP-HPLC. ESI-MS m/z calcd. for C₈H₁₃N₅O₁₂P₃ [M−H]⁻ 464.1, found [M−H]⁻ 464.1. HR ESI-MS m/z calcd. for C₈H₁₅N₅O₁₂P₃ [M+H]⁺ 465.9930,found [M+H]⁺ 465.9916 (Δm=−3.0 ppm).

Synthesis of pppGpG. The dinucleotide substrate pppGpG was synthesized by abortive in vitro transcription using T7 RNA polymerase (Huang, et al. (1998) Chem. Biol. 5:669-678; Kuzmine & Martin (2001) J. Mol. Biol. 305:559-566). The transcription template was the reverse oligonucleotide 5′-TAT AGT GAG TCG TAT TAT CCT ATA GTG AGT CGT ATT ACA GCG TGC GT-3′ (SEQ ID NO:43, initiation site for pppGpG synthesis underlined), together with the standard T7 promoter oligonucleotide disclosed herein. Using 5′-CCC TAT AGT GAG TCG TAT TAC AGC GTG CGT-3′ (SEQ ID NO:44) as template led to a smaller amount of desired dinucleotide product due to ‘slippage’ and therefore synthesis of a ladder of oligoguanosine nucleotides (Kuzmine & Martin (2001) supra). The in vitro transcription reaction was performed in the presence of GTP as the only nucleotide triphosphate. In a typical experiment, 200 pmol of the reverse oligonucleotide was annealed with 200 pmol of promoter oligonucleotide in 5 mM Tris, pH 7.5, followed by adjustment to final concentrations of 40 mM Tris, pH 8.0, 20 mM MgCl₂, 10 mM GTP, 10 mM DTT, and 2 mM spermidine in a total reaction volume of 200 μL. The transcription was initiated by the addition of T7 RNA polymerase and the sample was incubated at 37° C. for 4 hours. A solution of EDTA (25 μL of 0.5 M EDTA, pH 8.0) was added to the turbid reaction mixture, and the sample was vortexed until the magnesium pyrophosphate precipitate was completely dissolved. The clear solution was then extracted with 200 μL of 25:24:1 phenol/chloroform/isoamyl alcohol, and the products were precipitated by the addition of 1.2 mL of acetone. Turbidity appeared immediately; the mixture was frozen at −80° C. for 30 minutes, and the precipitate was recovered by centrifugation at 16000 g and 4° C. for 50 minutes. The supernatant was removed. The white pellet was dried under vacuum and redissolved in 15 μL H₂O. The desired dinucleotide product was isolated from the crude mixture by RP-HPLC on a Beckman Ultrasphere ODS 5U column (4.6×150 mm) with a gradient of 0-10% B in A over 15 minutes at 45° C. (A: 100 mM aqueous triethylammonium acetate (TEAA), B: CH₃CN; UV detection at 260 nm). The product containing fractions were combined and evaporated to dryness. To remove excess TEAA, the product was dissolved in 250 μL H₂O and evaporated four times. Finally, the product was dissolved in 30 μL H₂O, and the concentration was determined by UV absorbance (ε₂₆₀ 23400 L·mol⁻¹·cm⁻¹). Yield: 120 nmol pppGpG. ESI-MS calcd. for C₂₀H₂₈N₁₀O₂₁P₄ [M−H]⁻ 867.4, found [M−H+Et₃N]⁻ 968.6.

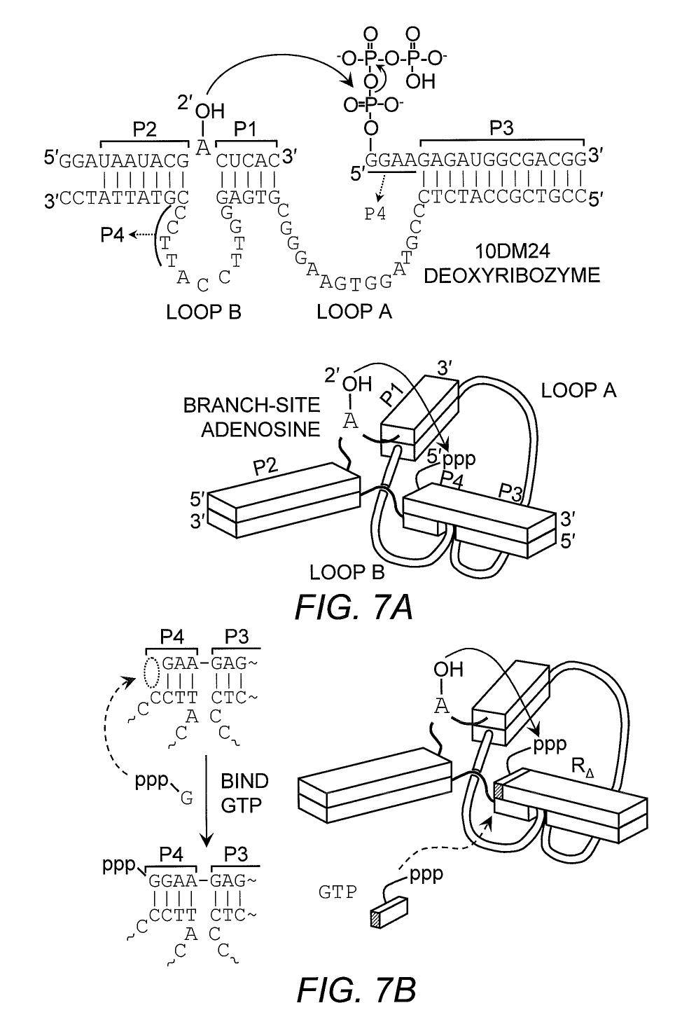

Use of Free NTPs as Substrates. It was determined whether 10DM24 could catalyze ligation with free GTP as a substrate (see FIG. 1B), thereby transferring guanosine 5′-monophosphate (GMP) to the branch-site adenosine 2′-hydroxyl group. When RΔ, an oligoribonucleotide cofactor that corresponds to all of the remaining nucleotides of R, was added to 10DM24 along with GTP, ligation was efficient (FIG. 8; 94% yield in 5 hours and k_{obs} 0.034 min⁻¹ under the standard incubation conditions of 1 mM GTP and 40 mM MgCl₂ at pH 9.0, 37° C.). The RNA product with the single added guanosine at the branch-site adenosine was PAGE-purified and the identity confirmed by partial alkaline hydrolysis (50 mM NaHCO₃ at 95° C. for 5 minutes) and MALDI mass spectrometry (m/z calcd. 5433, found 5437±5).

From a plot of k_{obs} versus [GTP], the K_{d,app} for GTP was found to be >1 mM. The k_{obs} increased eight-fold to 0.26 min⁻¹ under enhanced incubation conditions of 10 mM GTP and 150 mM MgCl₂ at pH 9.0, 37° C. (94% yield in 3 hours). Moreover, the ligation reaction of GTP with the 2′-hydroxyl group of the branch-site adenosine in the substrate RNA (L) required the presence of the cofactor RNA, RΔ. Different phosphorylation states of RΔ including nonphosphorylated (^{HO}RΔ), 5′-monophosphorylated (^{p}RΔ), and 5′-triphosphorylated ^{ppp}RΔ) were tolerated. However, the highest ligation efficiency was observed with ^{p}RΔ.

The generality of the ligation reaction using other NTP substrates in place of GTP was also determined. The analogous reaction with the full-length R oligonucleotide as substrate proceeded well when a 5′-terminal G was present (k_{obs} 0.51 min⁻¹ for 5′-AppG), with only three-fold reduced rate with 5′-AppA (k_{obs} 0.18 min⁻¹). The RNA substrate with 5′-AppC reacted 20-fold more slowly than 5′-AppG, but still gave high yield (k_{obs} 0.024 min⁻¹; 89% in 3 hours), whereas the yield with 5′-AppU was very poor (k_{obs} 0.002 min⁻¹; 9% in 3 hours). It is noted that in all cases, the corresponding deoxyribozyme nucleotide was changed to maintain Watson-Crick complementarity.

Focus was then placed on the purine NTPs (GTP, ATP) and their derivatives. When 1 mM ATP was provided as a small molecule substrate in place of GTP using the original 10DM24 sequence and RΔ, no reaction was observed (<1% in 5 hours). However, when the corresponding deoxyribozyme nucleotide was changed from C→T, substantial ligation was observed with ATP (33% yield in 5 hours and k_{obs} 0.0008 min⁻¹ under standard conditions; FIG. 8) but no longer with GTP (<1% in 5 hours). Furthermore, the k_{obs} increased sixteen-fold to 0.013 min⁻¹ under enhanced conditions with 10 mM ATP (82% yield in 3 hours). These data were as expected for a Watson-Crick base pair between the deoxyribozyme and the NTP substrate.

The number of hydrogen bonds between the NTP substrate and deoxyribozyme were also varied to determine whether there was any influence on the efficiency of the ligation reaction. Indeed, the ligation yield and rate increased when 2,6-diaminopurine ribonucleoside triphosphate (DTP) rather than ATP was paired with T in the deoxyribozyme (FIG. 2; 68% in 5 hours and k_{obs} 0.0032 min⁻¹ under standard conditions; 90% in 3 hours and k_{obs} 0.027 min⁻¹ under enhanced conditions). In contrast, when the original C in the deoxyribozyme was retained and inosine triphosphate (ITP) rather than GTP was provided as the substrate, a decrease in activity was observed (25% in 5 hours and k_{obs} 0.0007 min⁻¹ under standard conditions; 84% in 3 hours and k_{obs} 0.014 min⁻¹ under enhanced conditions). Therefore, three hydrogen bonds (GTP, DTP) rather than two hydrogen bonds (ITP, ATP) led to better activity (solid versus dashed lines in plot of FIG. 2). See also Table 6. Unexpectedly, replacing the adenine nucleobase of the substrate with 2-aminopurine led to a ten-fold decrease in k_{obs}, even though both purine derivatives can form two hydrogen bonds with T in the deoxyribozyme. Therefore, in terms of contribution to NTP substrate binding, the hydrogen bond facing the major groove was more important than the hydrogen bond facing the minor groove. For practical reasons, these experiments were performed with the 2′-deoxy-NTPs (i.e., dATP and d2AP-TP, where 2AP is 2-aminopurine). Because dATP is almost as efficient a substrate as ATP, the 2′-deoxy modification of d2AP-TP is not responsible for its poor reactivity as a substrate relative to dATP.

TABLE 6

| | Standard Incubation | | | Enhanced Incubation | | | |
|---|---|---|---|---|---|---|---|
| NTP | Conditions^{†} | | | Conditions^{‡} | | | k_{obs,enh}/ |
| NTP* | k_{obs} (min⁻¹) | %, 5 h | Kᵣₑₗ^{[a]} | k_{obs} (min⁻¹) | %, 3 h | Kᵣₑₗ^{[a]} | k_{obs,std} |
| GTP | 0.034 | 94 | (1) | 0.262 | 94 | (1) | 8 |
| dGTP | 0.020 | 88 | 0.60 | 0.081 | 90 | 0.31 | 4 |
| ddGTP^{[b]} | 0.013 | 85 | 0.38 | n.d. | n.d. | — | — |
| ITP | 0.0007 | 25 | 0.02 | 0.014 | 84 | 0.05 | 21 |
| d2AP-TP | <0.0001^{[c]} | <1 | <0.0003 | 0.0008 | 17 | 0.003 | >8 |
| G^{clv}-TP | <0.0001 | <1 | <0.0003 | 0.0003 | 6 | 0.001 | >3 |
| G^{acv}-TP | <0.0001 | <1 | <0.0003 | 0.0007 | 14 | 0.003 | >7 |
| ATP | 0.0008 | 33 | (1) | 0.013 | 82 | (1) | 16 |
| dATP^{[x]} | 0.0004 | 17 | 0.50 | 0.007 | 65 | 0.56 | 19 |
| ddATP^{[x]} | 0.0007 | 25 | 0.87 | n.d. | n.d. | — | — |
| ara-ATP | 0.0004 | 13 | 0.50 | 0.005 | 44 | 0.41 | 13 |
| DTP | 0.0032 | 68 | 4.0 | 0.027 | 90 | 2.1 | 8 |
| d2AP-TP | <0.0001^{[c]} | <1 | <0.1 | 0.0008 | 17 | 0.06 | >8 |

NTP = ATP or GTP;
NTP* = modified nucleotide triphosphate of GTP or ATP series,
^{†}1 mM NTP/NTP*, 40 mM MgCl₂, 100 mM CHES, pH 9.0, 150 mM NaCl, 2 mM KCl, 37° C.;
^{‡}10 mM NTP/NTP*, 150 mM MgCl₂, 100 mM CHES, pH 9.0, 150 mM NaCl, 2 mM KCl, 37° C.;
n.d. = not determined;
^{[a]}kᵣₑₗ = k_{obs,NTP*}/k_{obs,NTP},
^{[b]}standard conditions except with 50 mM CHES,
^{[c]}standard conditions with 5′-OH-RΔ.

The structural model for the 10DM24-catalyzed 2′,5′-RNA ligation reaction involving the original full-length R substrate indicates the presence of a Watson-Crick base pair at the second position of P4 (FIG. 7B). This model was investigated in more detail. The assays were performed according to established methods (Coppins & Silverman (2005) supra). The data were consistent with formation of a Watson-Crick base pair at the second position of P4. The RNA substrate with a G nucleotide at the second position was used promiscuously by all of the mismatched deoxyribozymes (i.e., the three 10DM24-C43X variants) with only a modest reduction in ligation rate. Nevertheless, the base-paired 10DM24 deoxyribozyme was still the most favorable combination (G-C base pair). For the RNA substrates with C or A at the second position, the Watson-Crick match was clearly preferred. Finally, for the RNA substrate with U at the second position, either a U-A base pair or a U-G wobble pair was favored.

In a separate assay, the overall requirement for a base pair at the second position of P4 was investigated in the context of the engineered NTP binding site in 10DM24. These data provide support for the binding model of the NTP substrate at the first position of P4, with the RΔ cofactor forming the remaining three base pairs of P4 (FIG. 7B). Only in the case of RΔ with 5′-G was substantial ligation activity observed with the mutant deoxyribozymes that do not allow for Watson-Crick base-pair formation with RΔ. Even so, the highest rate and yield were observed in the base-paired case. These observations are consistent with the promiscuity observed for G at the second position for ligation of the full-length R substrate. In the other cases of RΔ with 5′-C, 5′-A, or 5′-U, the Watson-Crick base pair at the second position of P4 was clearly favored.

The latter data, along with the Watson-Crick covariation involving the NTP substrate itself, provide support for the binding model depicted in FIG. 7B. To place this Watson-Crick binding mode in context, the other artificial aptamers and nucleic acid enzymes that interact with NTP substrates generally do so via non-Watson-Crick interactions (where the interaction mode is known), with μM to mM binding constants (Huang, et al. (1998) Chem. Biol. 5:669-678; Li, et al. (2000) Biochemistry 39:3106-3114; Li & Breaker (1999) Proc. Natl. Acad. Sci. USA 96:2746-2751; Wang, et al. (2002) Chem. Biol. 9:507-517). In contrast, the natural purine-binding riboswitches bind their cognate nucleobase via Watson-Crick interactions (Batey, et al. (2004) Nature 432:411-415; Serganov, et al. (2004) Chem. Biol. 11:1729-1741). In the latter cases, the nucleobase ligands are completely engulfed by the RNA, which enables quite low (nM) dissociation constants. A Watson-Crick binding mode is also observed for the preQ1 riboswitch, which has nM affinity for its ligand (Roth, et al. (2007) Nat. Struct. Mol. Biol. 14:308-317). It should be noted that not all biologically relevant interactions between RNA and substrates are high affinity; for example, the glmS riboswitch binds glucosamine 6-phosphate (GlcN6P) with K_{d,app} of merely 0.2 mM (Winkler, et al. (2004) Nature 428:281-286).

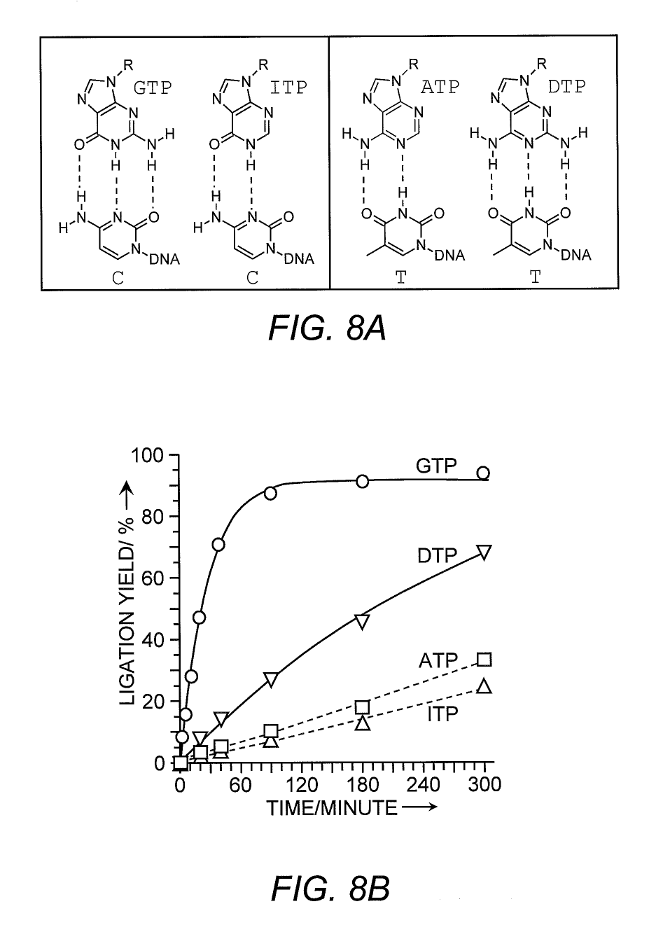

Nucleobase stacking interactions can contribute powerfully to macromolecular folding and binding processes (Hamuro, et al. (1997) J. Am. Chem. Soc. 119:10587-10593; Zhao & Moore (2002) J. Org. Chem. 67:3548-3554), particularly those involving nucleic acids (Hermann & Patel (2000) Science 287:820-825; Guckian, et al. (2000) J. Am. Chem. Soc. 122:2213-2222; Kool (2001) Annu. Rev. Biophys. Biomol. Struct. 30:1-22; Martin (1996) Chem. Rev. 96:3043-3064). The data herein establish that the small molecule NTP substrate of the 10DM24 deoxyribozyme binds at the 5′-terminal position of the P4 helix. In principle, the identity of the second P4 ribonucleotide could influence the NTP binding affinity by controlling the strength of stacking interactions with the NTP. To test this, the base pair that comprises the relevant RNA nucleotide and its deoxyribozyme counterpart were systematically altered. No clear pattern of ligation activity emerged, and in particular the more poorly stacking pyrimidine nucleotides did not lead to worse activity when placed at the second P4 position (G>U/C>A; FIG. 9). The phosphorylation state of the 5′-terminus of the RΔ cofactor could be varied (5′-monophosphate or 5′-OH) without altering the reactivity order G>U/C>A, although the 5′-OH—RΔ did lead to k_{obs} values that were up to four-fold lower (see Table 7). Thus, stacking interactions do not dominate binding affinity for the NTP.

TABLE 7

| 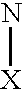 | k_{obs} (min⁻¹) 5̅′̅-̅p̅-̅R̅Δ̅ 5̅′̅-̅O̅H̅-̅R̅Δ̅ | $\frac{{K}_{obs , 5 ' - p - R Δ}}{{K}_{obs , 5 ' - OH - R Δ}}$ |
|---|---|---|
| 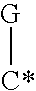 | 0.048 0.012 (0.034 (0.016) | 4.0(2.1) |
| 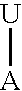 | 0.0056 0.0048 | 1.2 |
| 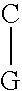 | 0.0095 0.0026 | 3.7 |
| 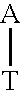 | 0.0012 0.0003 | 4.0 |

*For the parent 10DM24-substrate combination that has a G-C base pair at the second position of P4, the average values for k_{obs} are given in parentheses (n = 9 for 5′-p-RΔ; n = 3 for 5′-OH-RΔ).

Changes to the small-molecule NTP substrate were also evaluated to probe the role of the ribose ring, including potential effects of structural preorganization. Both 2′-deoxyGTP (dGTP) and 2′,3′-dideoxyGTP (ddGTP) were tolerated well, with no dimunition of yield and at most a three-fold decrease in k_{obs} relative to GTP. Similarly, arabino-ATP (which has the opposite 2′-configuration relative to ATP), dATP, and ddATP all had k_{obs} within two-fold of ATP itself. From these data, it was concluded that the deoxyribozyme did not require the 2′- or 3′-hydroxyl groups, nor did it directly contact either the 2′- or 3′-hydrogens of the ribose ring. It was additionally considered how perturbations in the structural preorganization of the substrate impact the ligation activity, using two substrate analogs. First, in place of GTP was used C2-C3-cleaved GTP (G^{clv}TP), which lacks the C2-C3 bond of the ribose ring but has the same number of heavy (non-hydrogen) atoms.

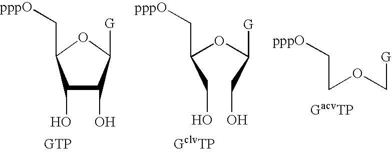

Second, in place of GTP was used acyclovir triphosphate (G^{acv}TP), where acyclovir is the guanosine analog that lacks both the C2 and C3 carbons and hydroxyl groups of the ribose ring. For both G^{clv}TP and G^{acv}TP, only a very small amount of ligation activity was observed; k_{obs} was diminished relative to GTP by approximately 1000-fold (G^{clv}TP) or 300-fold (G^{acv}TP) (see Table 6). The products were isolated by PAGE; all had the expected connectivity, as confirmed by partial alkaline hydrolysis. By design, the nucleobase and triphosphate (i.e., recognition and reactive) moieties of G^{clv}TP and G^{acv}TP were not structurally constrained by the five-membered ribose ring that was present within GTP itself. Therefore, the poor reactivities of these two modified substrates demonstrated that the preorganization enforced by the ribose ring of GTP contributed substantially to the efficiency of the deoxyribozyme-catalyzed ligation reaction.

All previous deoxyribozyme-mediated ligation reactions using two RNA oligonucleotide substrates had displayed only single-turnover ligation behavior, which was attributed to product inhibition (similar to natural protein enzymes that ligate nucleic acids; Flynn-Charlebois, et al. (2003) J. Am. Chem. Soc. 125:2444-2454). In contrast, upon 10DM24-catalyzed reaction of the oligoribonucleotide 2′-hydroxyl group with the NTP substrate, the binding affinity of the RNA for the deoxyribozyme was not expected to increase substantially. Thus, the capability of the engineered 10DM24 deoxyribozyme to catalyze the multiple-turnover ligation of GTP was examined. The 10DM24 deoxyribozyme was the limiting reagent, with the RNA substrate that provides the branch-site adenosine (L) in 10-fold excess over the deoxyribozyme (L:E:RΔ=10:1:3). The reaction was performed under the standard incubation conditions described herein using 1 mM GTP and 40 mM MgCl₂ at 37° C. After 5 hours, the product yield was 50%, corresponding to 5 turnovers. Accordingly, with GTP as substrate, multiple-turnover behavior was shown using an RNA ligase deoxyribozyme.

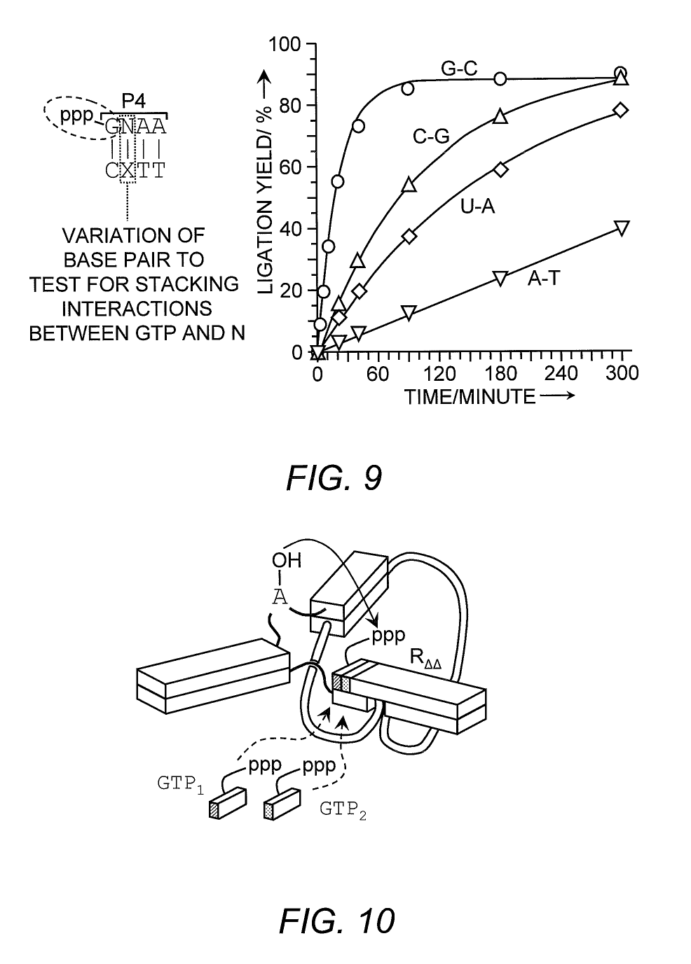

It was subsequently shown that a binding site for an NTP cofactor can be located adjacent to the substrate binding site. This was achieved by removing an additional nucleotide from the RΔ cofactor, forming the shorter RΔΔ cofactor which required two added nucleotides to reconstitute the complete P4 region (FIG. 10). The 10DM24-catalyzed ligation reaction of GTP in the presence of the two-nucleotide short cofactor RΔΔ was performed with the 5′-³²P-labeled RNA substrate (L) that provides the branch-site adenosine as the limiting reagent. For kinetic assays, the ratio of L:E:RΔΔ was 1:10:30 with 0.25 μM deoxyribozyme (E). For isolation of the reaction products, the ratio L:E:RΔΔ was 1:2:3 with 1.5 μM deoxyribozyme. The reaction was performed under the enhanced incubation conditions with 20 mM GTP and 150 mM MgCl₂ in 100 mM CHES, pH 9.0, 150 mM NaCl, and 2 mM KCl at 37° C. for up to 7 hours. The ligation reaction with GTP resulted in the formation of two reaction products. Both products were isolated and shown by partial alkaline hydrolysis to be branched with the connectivities A-G and A-GG (where A is the branch-site adenosine). Incubation with dGTP in place of GTP produced only the single-nucleotide addition product. When the first two nucleotides of P4 in the deoxyribozyme (nucleotides 43 and 44) were changed from CC→TT, ATP was accepted as both substrate and cofactor, because incubation with 20 mM ATP and 150 mM MgCl₂ in the presence of RΔΔ and the 10DM24 variant resulted in a small amount of the ATP ligation product.

Incubation at high pH for prolonged times resulted in partial random degradation of the RNA. Under the reaction conditions for ligation of GTP in the presence of RΔΔ, this random RNA degradation was <25% after 7 hours at pH 9.0 and 37° C.

It was contemplated that the new A-GG product was formed by initial templated but otherwise uncatalyzed synthesis of a GG dinucleotide (i.e., pppGpG) from two GTP molecules, followed by 10DM24-catalyzed branch formation using this dinucleotide. To demonstrate this, the purified A-G product was tested as a substrate for 10DM24-catalyzed ligation of GTP in the presence of the RΔΔ cofactor [(A-G):E:RΔΔ ˜1:10:30]. The reaction was performed under the enhanced incubation conditions of 20 mM GTP and 150 mM MgCl₂. After incubation at 37° C. for 3 hours, no new product formation was observed. This indicated that two GTP nucleotides could not be attached successively to the branch-site adenosine. The pppGpG dinucleotide was synthesized independently using T7 RNA polymerase (Huang, et al. (1998) supra) and led solely to the A-GG product. Although the pppGpG substrate had K_{d,app} of >1 mM with RΔΔ, similar to K_{d,app} for GTP with RΔ, the ligation reaction with pppGpG and RΔΔ had k_{obs} six-fold higher than for the analogous reaction with GTP and RΔ (Table 8).

TABLE 8

| Substrates | Mg⁺² | k_{obs} (min⁻¹) |
|---|---|---|
| 1 mM GTP + RΔ | 40 mM | 0.034 |
| 20 mM GTP + RΔΔ | 150 mM | 0.0008 |
| 1 mM pppGpG + RΔΔ | 40 mM | 0.19 |

Similar experiments were performed using the mutant 10DM24 deoxyribozyme that has CTTT rather than CCTT in the P4 region (FIG. 11). For the DNA-catalyzed ligation of pppGpG, the 5′-³²P-labeled RNA with the branch-site adenosine (L) was incubated with the 10DM24 deoxyribozyme (E) and the cofactor RΔΔ in the ratio of L:E:RΔΔ=1:10:30 in the presence of 1 mM pppGpG and 40 mM MgCl₂ in 100 mM CHES pH 9.0, 150 mM NaCl, and 2 mM KCl at 37° C. The analysis of the ligation reaction was performed as described herein. The ligation with pppGpG was efficient in the presence of RΔΔ; in contrast, in the absence of RΔΔ no product formation was observed. This is consistent with the model that pppGpG and the first two nucleotides of RΔΔ together reconstitute the P4 helix. When the ligation reaction with pppGpG was tested in the presence of RΔ instead of RΔΔ, the ligation product A-GG was formed, but with a 10-fold decrease in k_{obs} (squares in FIG. 11). Because RΔ provides an extraneous G nucleotide when combined with pppGpG (see legend of FIG. 11), some disruption in activity is perhaps anticipated. Mutation of the second deoxyribozyme nucleotide in P4 from C→T resulted in a 130-fold decrease in k_{obs} (inverted triangles in FIG. 11) No product formation was observed when the first DNA nucleotide in P4 (nucleotide 44) was changed from C→T (triangles in FIG. 11). This is consistent with the observation that GTP was also not a substrate for the 10DM24-C44T mutant deoxyribozyme.

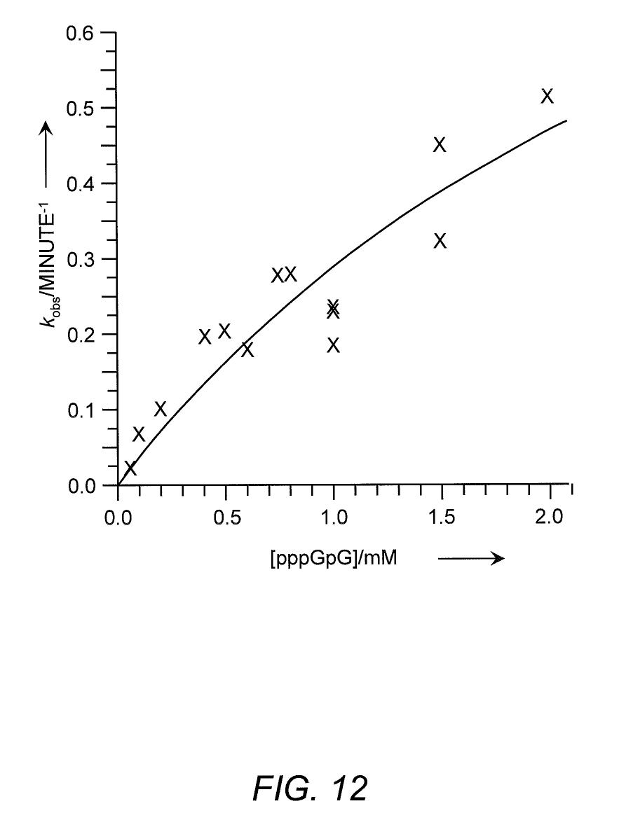

The ligation reaction of pppGpG was also performed using a variety of pppGpG concentrations with the parent 10DM24 deoxyribozyme in the presence of RΔΔ and 40 mM MgCl₂. In FIG. 12, the k_{obs} values are plotted versus [pppGpG] and fit to k_{obs}=kₘₐₓ·[pppGpG]/(K_{d,app}+[pppGpG]). The K_{d,app} for pppGpG was >1 mM.

What is claimed is:

1. A method for labeling a target ribonucleic acid (RNA) molecule comprising contacting a target RNA with the tagging RNA in the presence of a deoxyribozyme that is complementary to at least a portion of the target RNA and at least a portion of the tagging RNA so that the tagging RNA is site-specifically attached to the target RNA, wherein the tagging RNA is coupled to a label prior to or after attachment to the target RNA thereby labeling the target RNA molecule.

2. The method of claim 1, further comprising contacting the labeled target RNA with a second deoxyribozyme to remove one or more tagging RNA nucleotides.

3. A method for labeling a target RNA molecule comprising contacting a target RNA with at least one phosphorylated nucleotide in the presence of a cofactor and deoxyribozyme that is complementary to at least a portion of the target RNA, the phosphorylated nucleotide and at least a portion of the cofactor so that the phosphorylated nucleotide is site-specifically attached to the target RNA.

4. The method of claim 3, wherein the phosphorylated nucleotide is coupled with a label prior to being attached to the target RNA.

5. The method of claim 3, wherein the phosphorylated nucleotide is coupled with a label after being attached to the target RNA.


SEQUENCE LISTING (44 sequences)

SEQ ID NO: 1 - DNA, 11 bp, Artificial sequence
  Synthetic oligonucleotide
ggttccattgc

SEQ ID NO: 2 - DNA, 15 bp, Artificial sequence
  Synthetic oligonucleotide
ccgtaggtgaagggc

SEQ ID NO: 3 - RNA, 160 nt, Artificial sequence
  Synthetic RNA
ggaauugcgggaaaggggucaacagccguucaguaccaagucucaggggaaacuuugagauggccuugcaaaggguaugguaauaagcugacggacaugguccuaaccacgcagccaaguccuaagucaacagaucuucuguugauauggaugcaguuca

SEQ ID NO: 4 - RNA, 19 nt, Artificial sequence
  Synthetic oligonucleotide
gcaagagauggugauggga

SEQ ID NO: 5 - DNA, 33 bp, Artificial sequence
  Synthetic oligonucleotide
tcccatcaccaggctagctacaacgactcttgc

SEQ ID NO: 6 - DNA, 24 bp, Artificial sequence
  Synthetic oligonucleotide
agcgatccggaacggcacccatgt

SEQ ID NO: 7 - DNA, 15 bp, Artificial sequence
  Synthetic oligonucleotide
gaagtagcgccgccg

SEQ ID NO: 8 - DNA, 15 bp, Artificial sequence
  Synthetic oligonucleotide
ggctagctacaacga

SEQ ID NO: 9 - DNA, 13 bp, Artificial sequence
  Synthetic oligonucleotide
ccgagccggacga

SEQ ID NO: 10 - DNA, 25 bp, Artificial sequence
  Synthetic oligonucleotide
aacggggctgtgcggctaggaagta

SEQ ID NO: 11 - DNA, 14 bp, Artificial sequence
  Synthetic oligonucleotide
ccgagccggtcgaa

SEQ ID NO: 12 - DNA, 54 bp, Artificial sequence
  Synthetic oligonucleotide
ccgtcgccatctcccgtaggtgaagggcgtgagggttccattcccgtattatcc

SEQ ID NO: 13 - RNA, 16 nt, Artificial sequence
  Synthetic oligonucleotide
ggauaauacgacucac

SEQ ID NO: 14 - RNA, 17 nt, Artificial sequence
  Synthetic oligonculeotide
ggaagagauggcgacgg

SEQ ID NO: 15 - RNA, 21 nt, Artificial sequence
  Synthetic oligonucleotide
gcaaaagagauggugauggga

SEQ ID NO: 16 - DNA, 46 bp, Artificial sequence
  Synthetic oligonucleotide
tcccatcaccatctcttgctatagtgagtcgtattacagcgtgcgt

SEQ ID NO: 17 - DNA, 27 bp, Artificial sequence
  Synthetic oligonucleotide
acgcacgctgtaatacgactcactata

SEQ ID NO: 18 - RNA, 20 nt, Artificial Sequence
  Synthetic oligonucleotide
ggauaauacgacucacuaua

SEQ ID NO: 19 - RNA, 20 nt, Artificial sequence
  Synthetic oligonucleotide
ggacggcguaaucuguuaua

SEQ ID NO: 20 - RNA, 20 nt, Artificial sequence
  Synthetic oligonucleotide
ggagccgcauaagacauaua

SEQ ID NO: 21 - RNA, 20 nt, Artificial sequence
  Synthetic oligonucleotide
ggaauuaugcagaguguaua

SEQ ID NO: 22 - DNA, 27 bp, Artificial sequence
  Synthetic oligonucleotide
acgcacgctgtaatacgactcactatt

SEQ ID NO: 23 - DNA, 54 bp, Artificial sequence
  Synthetic oligonucleotide
ccgtcgccatctcccgtaggtgaagggcgtgagggttccattctcgtattatcc

SEQ ID NO: 24 - DNA, 54 bp, Artificial sequence
  Synthetic oligonucleotide
ccgtcgccatctcccgtaggtgaagggcgtgagggttccattcacgtattatcc

SEQ ID NO: 25 - DNA, 54 bp, Artificial sequence
  Synthetic oligonucleotide
ccgtcgccatctcccgtaggtgaagggcgtgagggttccattcgcgtattatcc

SEQ ID NO: 26 - DNA, 54 bp, Artificial sequence
  Synthetic oligonucleotide
ccgtcgccatctcccgtaggtgaagggcgtgagggttccatttccgtattatcc

SEQ ID NO: 27 - DNA, 54 bp, Artificial sequence
  Synthetic oligonucleotide
ccgtcgccatctcccgtaggtgaagggcgtgagggttccattaccgtattatcc

SEQ ID NO: 28 - DNA, 54 bp, Artificial Sequence
  Synthetic oligonucleotide
ccgtcgccatctcccgtaggtgaagggcgtgagggttccattgccgtattatcc

SEQ ID NO: 29 - DNA, 54 bp, Artificial Sequence
  Synthetic oligonucleotide
ccgtcgccatctcccgtaggtgaagggcgtgagggttccattgtcgtattatcc

SEQ ID NO: 30 - DNA, 54 bp, Artificial Sequence
  Synthetic oligonucleotide
ccgtcgccatctcccgtaggtgaagggcgtgagggttccattatcgtattatcc

SEQ ID NO: 31 - DNA, 54 bp, Artificial Sequence
  Synthetic oligonucleotide
ccgtcgccatctcccgtaggtgaagggcgtgagggttccattttcgtattatcc

SEQ ID NO: 32 - RNA, 17 nt, Artificial Sequence
  Synthetic oligonucleotide
agaagagauggcgacgg

SEQ ID NO: 33 - RNA, 17 nt, Artificial Sequence
  Synthetic oligonucleotide
ugaagagauggcgacgg

SEQ ID NO: 34 - RNA, 17 nt, Artificial sequence
  Synthetic oligonucleotide
cgaagagauggcgacgg

SEQ ID NO: 35 - RNA, 17 nt, Artificial Sequence
  Synhetic oligonucleotide
gaaagagauggcgacgg

SEQ ID NO: 36 - RNA, 17 nt, Artificial Sequence
  Synthetic oligonucleotide
guaagagauggcgacgg

SEQ ID NO: 37 - RNA, 17 nt, Artificial Sequence
  Synthetic oligonucleotide
gcaagagauggcgacgg

SEQ ID NO: 38 - RNA, 16 nt, Artificial Sequence
  Synthetic oligonucleotide
gaagagauggcgacgg

SEQ ID NO: 39 - RNA, 16 nt, Artificial Sequence
  Synthetic oligonucleotide
aaagagauggcgacgg

SEQ ID NO: 40 - RNA, 16 nt, Artificial Sequence
  Synthetic oligonucleotide
uaagagauggcgacgg

SEQ ID NO: 41 - RNA, 16 nt, Artificial sequence
  Synthetic oligonucleotide
caagagauggcgacgg

SEQ ID NO: 42 - RNA, 15 nt, Artificial Sequence
  Synthetic oligonucleotide
aagagauggcgacgg

SEQ ID NO: 43 - DNA, 47 bp, Artificial Sequence
  Synthetic oligonucleotide
tatagtgagtcgtattatcctatagtgagtcgtattacagcgtgcgt

SEQ ID NO: 44 - DNA, 30 bp, Artificial Sequence
  Synthetic oligonucleotide
ccctatagtgagtcgtattacagcgtgcgt